(19) United States
(12) Patent Application Publication
(10) Pub. No.: US 2010/0215632 A1
(43) Pub. Date: Aug. 26, 2010

(54) BIOMARKERS OF SIRTUIN ACTIVITY AND METHODS OF USE THEREOF

(75) Inventors: Olivier Boss, Boston, MA (US); Siva Worcester, Worcester, MA (US); Andre Iffland, Cambridge, MA (US); Jesse Smith, Waltham, MA (US); Jill Milne, Brookline, MA (US); Michael Jirousek, Cambridge, MA (US)

Correspondence Address: ROPES & GRAY LLP, PATENT DOCKETING 39/41, ONE INTERNATIONAL PLACE, BOSTON, MA, 02110-2624, US

(73) Assignee: Sirtris Pharmaceuticals, Inc., Cambridge, MA (US)

(21) Appl. No.: 12/450,252

(22) PCT Filed: Mar. 19, 2008
(86) PCT No.: PCT/US2008/003605

§ 371 Date: Mar. 24, 2010

Related U.S. Application Data
(60) Provisional application No. 60/918,735, filed on Mar. 19, 2007.

(51) Int. Cl. A61K 38/43; C12Q 1/44; C12Q 1/68; C12Q 1/02

(52) U.S. Cl. 424/94.1; 435/19; 435/6; 435/29

(57) ABSTRACT

Provided are methods for monitoring sirtuin modulation in a subject, for example, during therapeutic treatment with a sirtuin modulating compound. The methods involve determining the expression level of one or more sirtuin biomarkers in a biological sample from the subject. Also provided are methods for identifying compounds that modulate the activity of a sirtuin protein using one or more sirtuin biomarkers.

### RELATED APPLICATIONS

This application claims the benefit of priority to U.S. Provisional Application No. 60/918,735, filed Mar. 19, 2007, which application is hereby incorporated by reference in its entirety.

### BACKGROUND

Aging is a major risk factor for a variety of major diseases including type 2 diabetes, cancers, cardiovascular, metabolic and neurodegenerative diseases. Manipulations that extend lifespan, such as restriction of caloric intake (Calorie Restriction), can prevent or delay these metabolic changes and confer resistance to many disease in a range of organisms (Curtis et al. Nature Reviews Drug Discovery, 2005, vol. 4, 569-580). More recently, specific genetic pathways that modulate aging in invertebrates and rodents have been identified (Kenyon, C. Cell 120, 2005, 449-460). Changes in single genes within these pathways can cause dramatic increase in lifespan and these long-live organisms are less susceptible to age-related disease. Many of the genes that regulate life span are evolutionarily conserved.

The Silent Information Regulator (SIR) family of genes (or sirtuins) represents a highly conserved group of genes present in the genomes of organisms ranging from archaebacteria to a variety of eukaryotes. The encoded SIR proteins are involved in diverse processes from regulation of gene silencing to DNA repair. The proteins encoded by members of the SIR gene family show high sequence conservation in a 250 amino acid core domain. A well-characterized gene in this family is S. cerevisiae SIR2, which is involved in silencing HM loci that contain information specifying yeast mating type, telomere position effects and cell aging. The yeast Sir2 protein belongs to a family of histone deacetylases. The Sir2 homolog, CobB, in Salmonella typhimurium, functions as an NAD (nicotinamide adenine dinucleotide)-dependent ADP-ribosyl transferase.

The Sir2 protein is a class III deacetylase which uses NAD as a cosubstrate. Unlike other deacetylases, many of which are involved in gene silencing, Sir2 is insensitive to class I and II histone deacetylase inhibitors like trichostatin A (TSA).

Deacetylation of acetyl-lysine by Sir2 is tightly coupled to NAD hydrolysis, producing nicotinamide and a novel acetyl-ADP ribose compound. The NAD-dependent deacetylase activity of Sir2 is essential for its functions which can connect its biological role with cellular metabolism in yeast. Mammalian Sir2 homologs have NAD-dependent histone deacetylase activity. Most information about Sir2 mediated functions comes from the studies in yeast.

Biochemical studies have shown that Sir2 can readily deacetylate the amino-terminal tails of histones H3 and H4, resulting in the formation of 1-O-acetyl-ADP-ribose and nicotinamide. Strains with additional copies of SIR2 display increased rDNA silencing and a 30% longer life span. It has recently been shown that additional copies of the C. elegans SIR2 homolog, sir-2.1, and the D. melanogaster dSir2 gene greatly extend life span in those organisms. This implies that the SIR2-dependent regulatory pathway for aging arose early in evolution and has been well conserved. Today, Sir2 genes are believed to have evolved to enhance an organism's health and stress resistance to increase its chance of surviving adversity.

Caloric restriction has been known for over 70 years to improve the health and extend the lifespan of mammals. Yeast life span, like that of metazoans, is also extended by interventions that resemble caloric restriction, such as low glucose. The discovery that both yeast and flies lacking the SIR2 gene do not live longer when calorically restricted provides evidence that SIR2 genes mediate the beneficial health effects of this diet. Moreover, mutations that reduce the activity of the yeast glucose-responsive cAMP (adenosine 3′S′-monophosphate)-dependent (PKA) pathway extend life span in wild type cells but not in mutant sir2 strains, demonstrating that SIR2 is likely to be a key downstream component of the caloric restriction pathway.

Recently, a number of small molecule activators and inhibitors of the SIR proteins have been reported (see e.g., U.S. Patent Application Publication Nos. 2005/0136537 and 2005/0096256 and PCT Publication Nos. WO 2005/002555 and WO 2005/002672) and a number of uses for these compounds have been identified. For example, small molecule activators of SIR proteins were shown to extend life span in yeast and cultured human cells as well as activate SIR protein activity in human cells (supra). Additionally, the small molecule SIR activators were shown to mimic calorie restriction and extend lifespan in Caenorhabditis elegans and Drosophila melanogaster (supra). Activators of the SIR proteins may therefore be useful for mimicking the effects of calorie restriction in eukaryotic cells and treating aging-related diseases such as stroke, cardiovascular disease, arthritis, high blood pressure, or Alzheimer's disease (supra). Additionally, it has been shown that resveratrol, butein, fisetin, piceatannol, and quercetin, small molecule activators of SIR proteins, promote fat mobilization in C. elegans, prevent fat accumulation in C. elegans, stimulate fat mobilization in mammalian cells, and inhibit adipogenesis in mammalian cells (see e.g., U.S. Patent Publication No. 2005/0171027 and PCT Publication No. WO 2005/065667). Similarly, nicotinamide, an inhibitor of SIR proteins, was shown to promote fat accumulation (supra). Additionally, resveratrol was shown to at least partially restore insulin sensitivity in insulin resistant cells (supra). Activators of SIR proteins may therefore also be useful for treating or preventing insulin resistance disorders and have been suggested for uses relating to reducing weight or preventing weight gain (supra).

The human ortholog of yeast Sir2 (silent mating type information regulation 2), SIRT1, is an NAD⁺-dependent deacetylase. The SIRT1 protein is localized in the nucleus and interacts with and deacetylates a large number of proteins.

Unfortunately, it is difficult to monitor in vivo effects of therapeutic agents that increase the activity of a sirtuin protein. Accordingly, a need exists for novel sirtuin biomarkers that may be used to determine sirtuin activity in vivo as well as monitor sirtuin modulation upon therapeutic intervention.

### SUMMARY

Provided herein are methods for determining sirtuin activity in a subject. Such methods may be used for diagnostic and prognostic applications. Also provided are methods for monitoring sirtuin modulation in a subject including, for example, during therapeutic treatment with a sirtuin modulating compound. Methods for identifying compounds that modulate the activity of a sirtuin protein are also provided.

In one aspect, the invention provides a method for detecting modulation of a sirtuin protein in a subject, comprising determining the expression level of one or more sirtuin biomarkers (examples shown in Table 1) in a biological sample from the subject wherein a change in the expression level of one or more sirtuin biomarkers as compared to a control indicates sirtuin modulation in the subject.

In certain embodiments, the sirtuin modulation may be sirtuin activation. Sirtuin activation may be beneficial when a subject has low levels of sirtuin activity or when an increase in sirtuin activity would be beneficial to the subject. For example, subjects suffering from a disease or disorder related to aging or stress, diabetes, obesity, a neurodegenerative disease, chemotherapeutic induced neuropathy, neuropathy associated with an ischemic event, an ocular disease or disorder, cardiovascular disease, a blood clotting disorder, inflammation, or flushing, may benefit from treatment with a sirtuin activating compound.

In certain embodiments, the sirtuin modulation may be sirtuin inhibition. Sirtuin inhibition may be beneficial when a subject has high levels of sirtuin activity or when a subject is need of decreased sirtuin activity. For example, subjects that require appetite stimulation or weight gain, may benefit from treatment with a sirtuin inhibiting compound.

In certain embodiments, the expression level of at least one, two, three, four, five, ten, or more, sirtuin biomarkers shown in Table 1 may be determined.

In certain embodiments, the expression level of at least one of the following sirtuin biomarkers are determined: MCP-1, BMP Receptor 1A, Smpd13a, CD14, ApoE, FAS, Transthyretin, FABP1 (liver), Acyl-CoA thioesterase 1, Acyl-CoA thioesterase 2, Aquaporin 4, Rrad, CXCL9, CCL8, Ppp1r3g, ApoA-I, ApoA-II, ApoB, or FGF21. In certain embodiments, the expression level of MCP-1 is determined. In certain embodiments, the expression level of FGF21 is determined.

In certain embodiments, the expression level of one or more biomarkers is determined by measuring the mRNA level of one or more sirtuin biomarkers. In certain embodiments, the mRNA level of one or more sirtuin biomarkers is measured using a microarray chip. In certain embodiments, the mRNA level of one or more sirtuin biomarker is measured using PCR, for example, quantitative real-time PCR.

In certain embodiments, the expression level of one or more biomarkers is determined by measuring the protein level of one or more sirtuin biomarkers. In certain embodiments, the protein level of one or more sirtuin biomarkers is determined using an antibody (e.g., immunoblotting, radioimmunoassay, ELISA, etc), mass spectrometry, or gel electrophoresis.

In certain embodiments, the expression level of one or more biomarkers is determined by measuring the activity level of one or more sirtuin biomarkers.

In certain embodiments, a change in the expression level of the one or more sirtuin biomarkers, as compared to a control, is indicative of therapeutic sirtuin modulation in said subject. In certain embodiments, a decrease of MCP-1 expression level, as compared to a control, indicates sirtuin activation. In certain embodiments, an increase in FGF21 expression level, as compared to a control, indicates sirtuin activation.

In certain embodiments, the control may be an untreated individual, the subject prior to treatment, the subject at an earlier time point during treatment, or a database reference.

In certain embodiments, the biological sample may comprise blood, urine, serum, saliva, cells, tissue, and/or hair.

In certain embodiments, the subject may be a mammal, such as, for example, a human.

In another aspect, the invention provides a method for monitoring therapeutic treatment with a sirtuin modulator, comprising determining the expression level of one or more sirtuin biomarkers (examples shown in Table 1) in a biological sample from a subject being treated with a sirtuin modulator, wherein a change in the expression level of one or more sirtuin biomarkers, as compared to a control, indicates therapeutic sirtuin modulation in the subject.

In certain embodiments, the sirtuin modulator is a sirtuin activating compound. In certain embodiments, the sirtuin modulator is a sirtuin inhibiting compounds.

In certain embodiments, a decrease in the expression level of MCP-1 upon treatment with the sirtuin modulator indicates therapeutic sirtuin activation. In certain embodiments, an increase in the expression level of FGF21 upon treatment with the sirtuin modulator indicates therapeutic sirtuin activation.

In certain embodiments, the method may further comprise adjusting the dose of the sirtuin modulator administered to the subject, e.g., based on the expression level of one or more sirtuin biomarkers (examples shown in Table 1) in response to administration of the sirtuin modulator.

In another aspect, the invention provides a method for monitoring the progress of therapeutic treatment with a sirtuin modulator, comprising: (i) administering a sirtuin modulator to a subject, (ii) obtaining a biological sample from said subject, and (iii) determining the expression level of one or more sirtuin biomarkers (examples shown in Table 1) in the sample, wherein a change in the expression level of the one or more sirtuin biomarkers as compared to a control indicates therapeutic sirtuin modulation in said subject.

In certain embodiments, the sirtuin modulator may be administered to a subject at least twice over time and the expression level of one or more sirtuin biomarkers is determined at two or more time points during the course of administration.

In another aspect, the invention provides a method of identifying a subject that would benefit from treatment with a sirtuin modulating compound, comprising determining the expression level of one or more sirtuin biomarkers (examples shown in Table 1) in a biological sample from the subject, wherein an altered expression level of one or more sirtuin biomarkers as compared to a control indicates that a subject may benefit from treatment with a sirtuin modulating compound.

In certain embodiments, an altered expression level of one or more sirtuin biomarkers in a biological sample from a subject, as compared to a control, indicates therapeutic sirtuin activation. In certain embodiments, a decrease in the expression level of MCP-1, as compared to a control, indicates therapeutic sirtuin activation. In certain embodiments, an increase in the expression level of FGF21, as compared to a control, indicates therapeutic sirtuin activation.

In another aspect, the invention provides a method of evaluating a subject's risk of developing a sirtuin-mediated disease or disorder, comprising determining the expression level of one or more sirtuin biomarkers (examples shown in Table 1) in a biological sample from the subject, wherein an altered expression level of one or more sirtuin biomarkers, as compared to a control, indicates that the subject is at risk for developing a sirtuin-mediated disease or disorder.

In another aspect, the invention provides a method for treating a sirtuin-mediated disease or disorder in a subject, comprising: (i) administering a sirtuin modulating compound to the subject, and (ii) monitoring the expression level of one or more sirtuin biomarkers (examples shown in Table 1) over time to determine whether the course of treatment in the subject should be modified. In certain embodiments, the method further comprises determining the expression level of one or more sirtuin biomarkers prior to administration of the sirtuin modulating compound to identify a subject that would benefit from treatment with a sirtuin modulating compound.

In another aspect, the invention provides a method for identifying a compound that modulates a sirtuin protein, comprising: (i) contacting a cell that expresses a sirtuin protein with a test compound, and (ii) determining the expression level of one or more sirtuin biomarkers (examples shown in Table 1), wherein a change in the expression level of one or more sirtuin biomarkers in the presence of the test compound, as compared to a control, indicates that the test compound modulates the sirtuin protein. In certain embodiments, the cell may be a tissue culture cell. In certain embodiments, the cell may overexpress a sirtuin protein, such as, SIRT1.

In certain embodiments, a test compound that activates a sirtuin protein may be identified. In other embodiments, a test compound that inhibits a sirtuin protein may be identified. In certain embodiments, the test compound is a small molecule.

In certain embodiments, a modulator of a human sirtuin protein, such as SIRT1, may be identified.

In certain embodiments, the expression level of at least one, two, three, four, five, ten, or more, sirtuin biomarkers shown in Table 1 are determined.

In certain embodiments, the expression level of at least one of the following sirtuin biomarkers are determined: MCP-1, BMP Receptor 1A, Smpd13a, CD14, ApoE, FAS, Transthyretin, FABP1 (liver), Acyl-CoA thioesterase 1, Acyl-CoA thioesterase 2, Aquaporin 4, Rrad, CXCL9, CCL8, Ppp1r3g, ApoA-I, ApoA-II, ApoB, or FGF21. In certain embodiments, the expression level of MCP-1 is determined. In certain embodiments, the expression level of FGF21 is determined.

In certain embodiments, the expression level of one or more biomarkers is determined by measuring the mRNA, protein, and/or protein activity levels of one or more sirtuin biomarkers.

In certain embodiments, the cell is a mammalian cell, such as a human cell. The cell may be an isolated cell, suspended in culture, or may be present in a whole organism, such as a non-human organism.

In certain embodiments, a method for identifying a compound that modulates a sirtuin protein may further comprise one or more of the following: (i) preparing a quantity of the compound, or an analog thereof, (ii) conducting therapeutic profiling of the compound, or an analog thereof, for efficacy and toxicity in animals, (iii) formulating the compound, or analog thereof, in a pharmaceutical formulation, (iv) manufacturing a pharmaceutical preparation of a compound, or an analog thereof, having a suitable animal toxicity profile, or (v) marketing a pharmaceutical preparation of a compound, or an analog thereof, having a suitable animal toxicity profile to healthcare providers.

In another aspect, the invention provides a kit for detecting the expression level of a sirtuin biomarker, comprising at least one component for determining the expression level of one or more sirtuin biomarker (examples shown in Table 1) and at least one sirtuin modulating compound.

In certain embodiment, the component for determining the expression level of one or more sirtuin biomarkers is an antibody or an antigen-binding fragment thereof that binds to the sirtuin biomarker. In certain embodiment, the component for determining the expression level of one or more sirtuin biomarkers is a set of PCR primers that specifically amplify the sirtuin biomarker mRNA. In certain embodiment, the component for determining the expression level of one or more sirtuin biomarkers is a solid support comprising at least a fragment of the polynucleotide sequence encoding the sirtuin biomarker attached thereto (such as a microarray chip).

In certain embodiment, the kit further comprises one or more of the following: a detection label, buffer, or instructions for use, or a cell line that expresses a sirtuin protein.

In another aspect, the invention provides a method of determining the level of sirtuin activity in a biological sample, comprising determining the expression level of at least one sirtuin biomarker in the biological sample.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF THE FIGURES

FIG. 1 shows Table 1 which provides a list of sirtuin biomarkers identified from an in vitro study (using freshly isolated human WBC) and an in vivo study (using mouse models) demonstrating a more than two fold change in expression either up or down following treatment at the indicated time points. Human WBCs were treated with resveratrol or Compound 2; mouse models were treated with resveratrol or Compound 1. Biomarkers labeled with *** are those whose expression not only changed more than 2 fold up or down upon treatment with both compounds (i.e., Compound 1 and resveratrol with the mouse tissues or Compound 2 and resveratrol for the human WBCs) but also demonstrated the most robust or reproducible response. Preferred biomarkers either had the highest fold changes with low variability across experiments in the human WBC experiments or demonstrated the highest fold changes in tissues of interest in the mouse in vivo experiments. “up”: the expression level of the biomarker increased upon treatment; “down”: the expression level of the biomarker decreased upon treatment; “/”: there was no change; the 2 week time point was actually 16 days for the in vivo mouse study.

FIG. 2 shows FGF21 expression in 3 day liver samples of animals treated with resveratrol or Compound 1 at the indicated dose per day. The y axis illustrates FGF21 mRNA levels, and the x axis displays the results for mice treated with no drug (vehicle), resveratrol (1000 mg/kg), or Compound 1 (100 mg/kg).

FIG. 3 depicts FGF21 gene expression in response to overexpression of SIRT1 in tissue culture cells. The y axis illustrates FGF21 mRNA levels determined by quantitative PCR, and the x axis displays the results for cells transfected with a GFP-expressing control plasmid, 1 ug of SIRT1-expressing plasmid, or 5 ug of SIRT1-expressing plasmid.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The terms “comprise” and “comprising” are used in the inclusive, open sense, meaning that additional elements may be included.

The term “expression level,” when used in reference to a sirtuin biomarker, refers to a quantity reflected in or derivable from the biomarker's gene or protein expression data, such as gene transcript accumulation, protein accumulation, or a detectable biological activity of the biomarker.

The term “including” is used to mean “including but not limited to”. “Including” and “including but not limited to” are used interchangeably.

The term “mammal” is known in the art, and exemplary mammals include humans, primates, livestock animals (including bovines, porcines, etc.), companion animals (e.g., canines, felines, etc.) and rodents (e.g., mice and rats).

The term “modulate” or “modulation,” when used in reference to the activity of a sirtuin protein, refers to the up regulation (e.g., activation or stimulation), down regulation (e.g., inhibition or suppression), or other change in a quality of at least one activity of a sirtuin protein.

“Sirtuin-activating compound” refers to a compound that increases the level of a sirtuin protein and/or increases at least one activity of a sirtuin protein. A sirtuin-activating compound refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation of a sirtuin substrate (e.g., deacetylation of an acetylated polypeptide such as, for example, a histone or p53), extending lifespan, increasing genomic stability, silencing transcription, controlling segregation of oxidized proteins between mother and daughter cells, or modulating the expression level of at least one sirtuin biomarker (e.g., a gene shown in Table 1, such as, for example, MCP-1). Exemplary sirtuin activating compounds include flavones, stilbenes, flavanones, isoflavanones, catechins, chalcones, tannins and anthocyanidins. Exemplary stilbenes include hydroxystilbenes, such as trihydroxystilbenes, e.g., 3,5,4′-trihydroxystilbene (“resveratrol”). Resveratrol is also known as 3,4′,5-stilbenetriol. Tetrahydroxystilbenes, e.g., piceatannol, are also encompassed. Hydroxychalones including trihydroxychalones, such as isoliquiritigenin, and tetrahydroxychalones, such as butein, can also be used. Hydroxyflavones including tetrahydroxyflavones, such as fisetin, and pentahydroxyflavones, such as quercetin, can also be used. Other sirtuin activating compounds are described in U.S. Patent Application Publication No. 2005/0096256 and PCT Application Nos. PCT/US06/002092, PCT/US06/007746, PCT/US06/007744, PCT/US06/007745, PCT/US06/007778, PCT/US06/007656, PCT/US06/007655 and PCT/US06/007773.

“Sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation of a sirtuin substrate (e.g., deacetylation of an acetylated polypeptide such as, for example, a histone or p53), extending lifespan, increasing genomic stability, silencing transcription, controlling segregation of oxidized proteins between mother and daughter cells, or modulating the expression level of at least one sirtuin biomarker (e.g., a gene shown in Table 1, such as, for example, MCP-1). Exemplary sirtuin inhibitors include, for example, sirtinol and analogs thereof (see e.g., Napper et al., J. Med. Chem. 48: 8045-54 (2005)), nicotinamide (NAD⁺) and suramin and analogs thereof. Other sirtuin inhibiting compounds are described in U.S. Patent Application Publication No. 2005/0096256, PCT Publication No. WO2005/002527, and PCT Application Nos. PCT/US06/007746, PCT/US06/007744, PCT/US06/007745, PCT/US06/007778, PCT/US06/007656, PCT/US06/007655, PCT/US06/007773 and PCT/US06/007742.

“Sirtuin-modulating compound” refers to a compound that may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

### 2. Diagnostic and Therapeutic Methods

Provided herein are sirtuin biomarkers and methods of using sirtuin biomarkers for a wide variety of application including, for example, diagnostic applications, therapeutic monitoring applications and drug screening assays. The methods described herein involve determining the level of one or more sirtuin biomarkers in a biological sample. Such biomarkers are indicative of sirtuin activity in the biological sample or the subject from which the biological sample was obtained. In certain embodiments, the methods may involve determining the level of one sirtuin biomarker in the biological sample. In other embodiments, the methods may involve determining the level of two or more sirtuin biomarkers in a sample, such as, for example, the level of two, three, four, five, six, seven, eight, nine, ten, fifteen, twenty, twenty-five, fifty, or 100, or more sirtuin biomarkers in a sample.

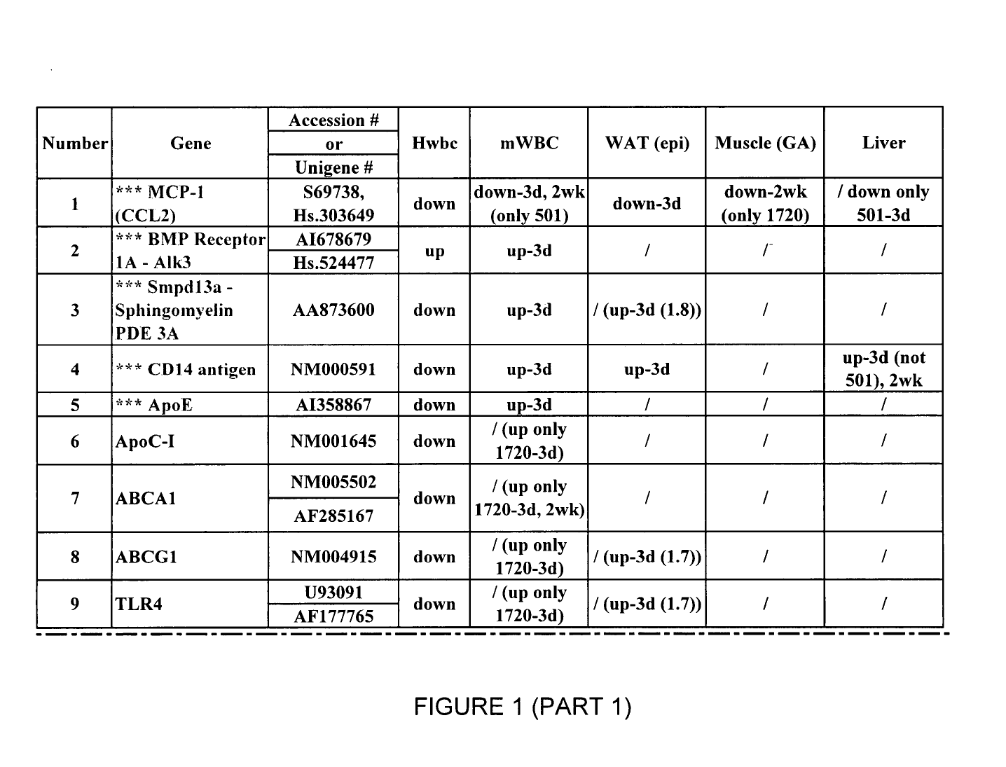

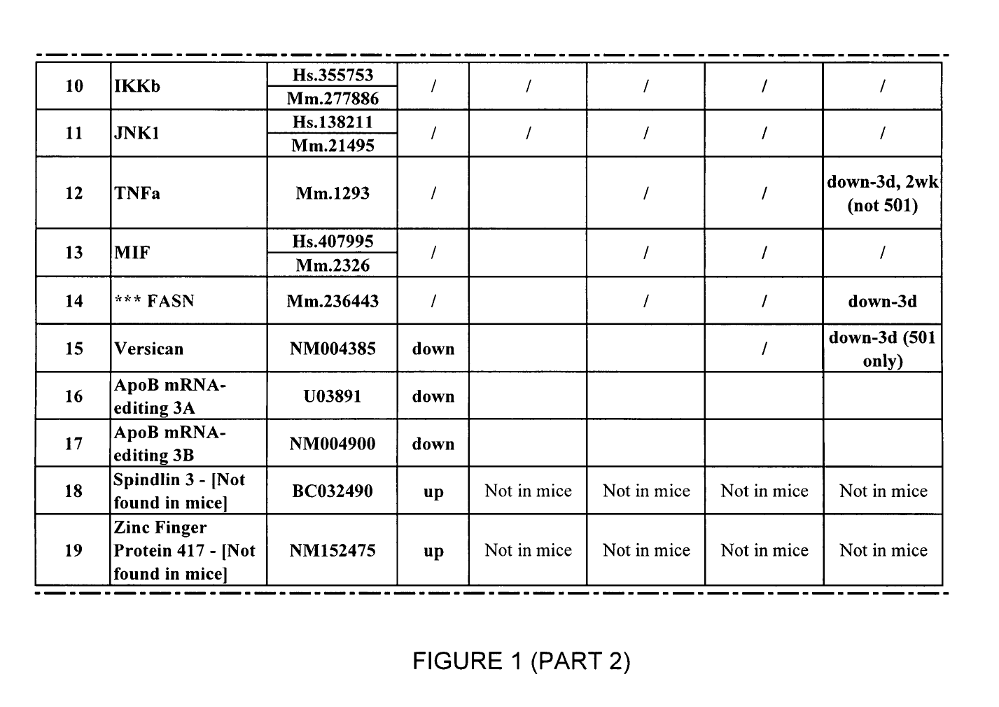

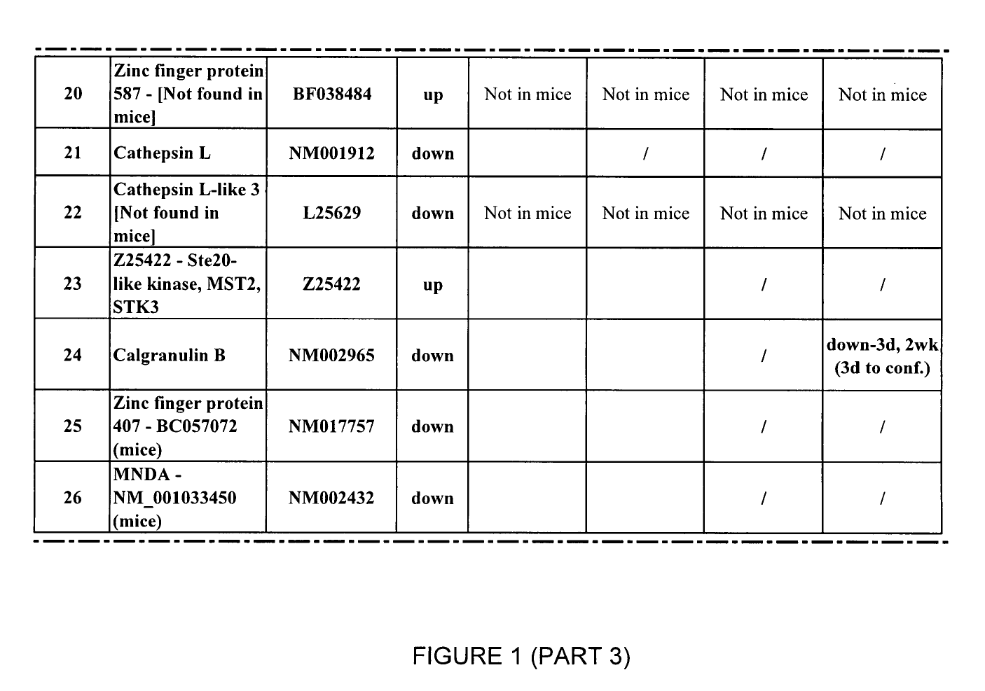

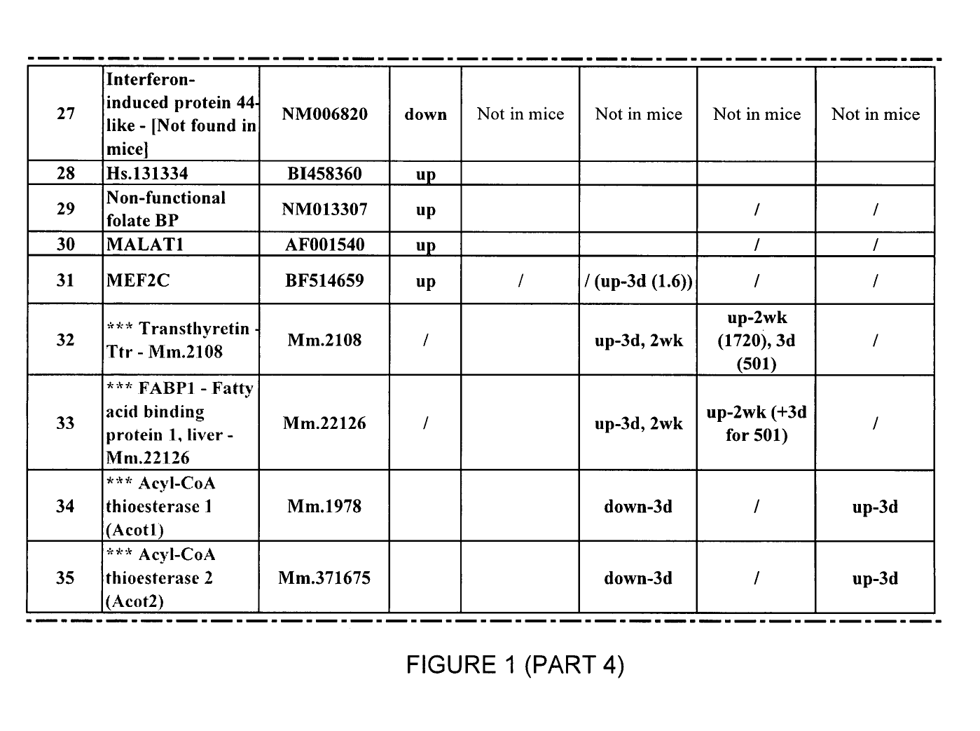

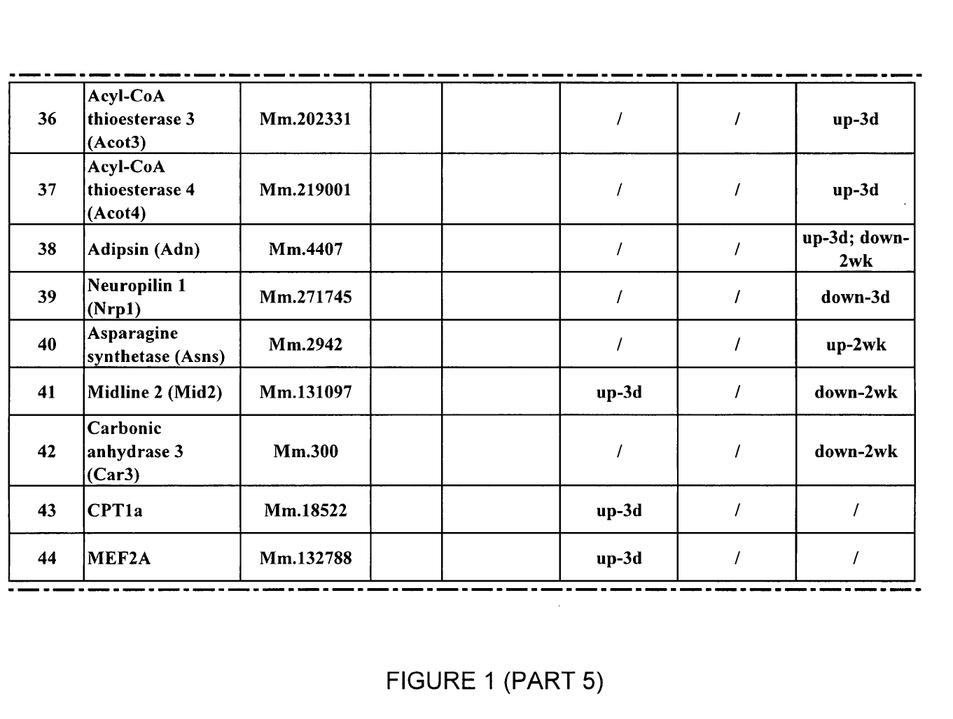

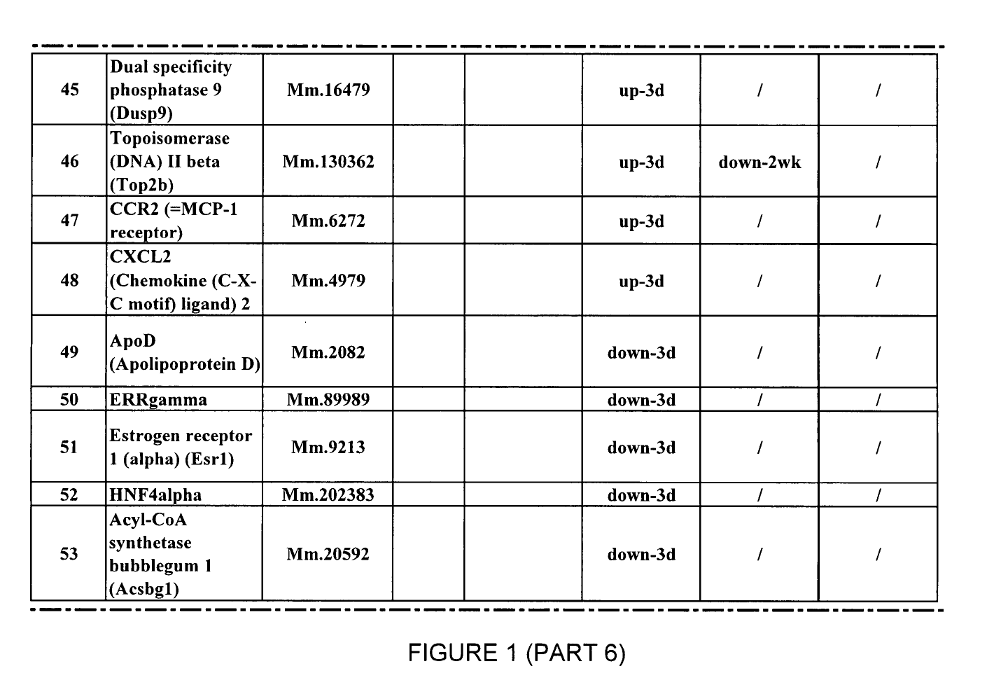

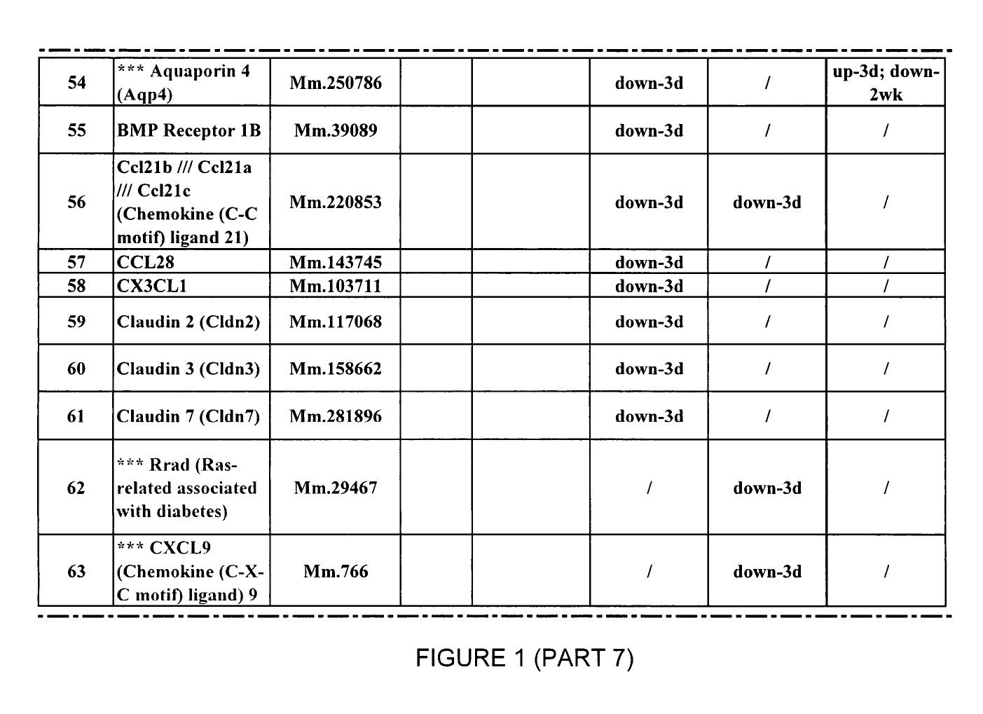

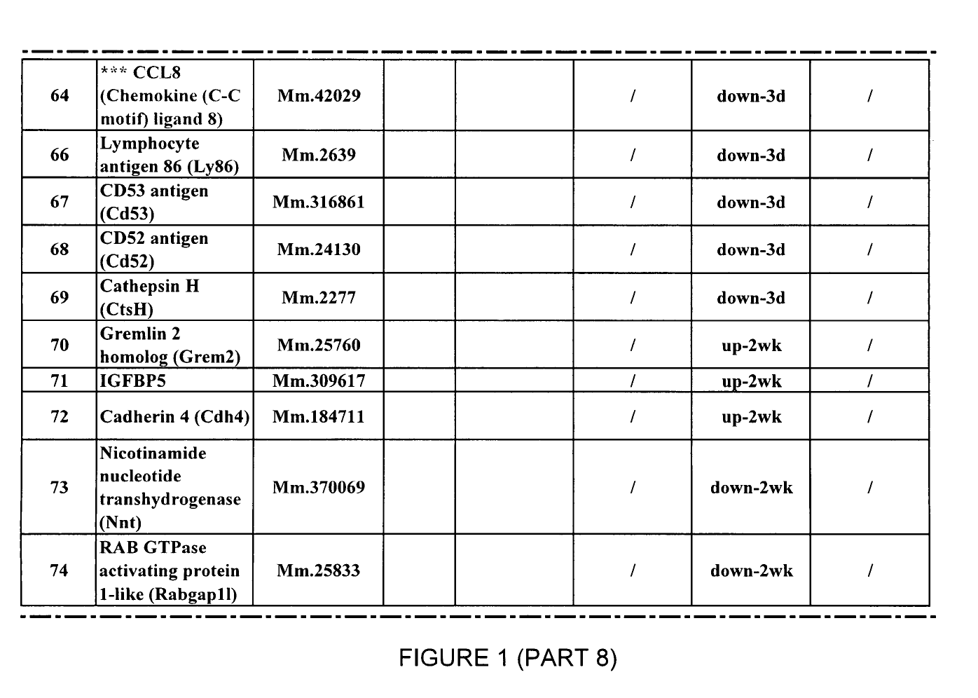

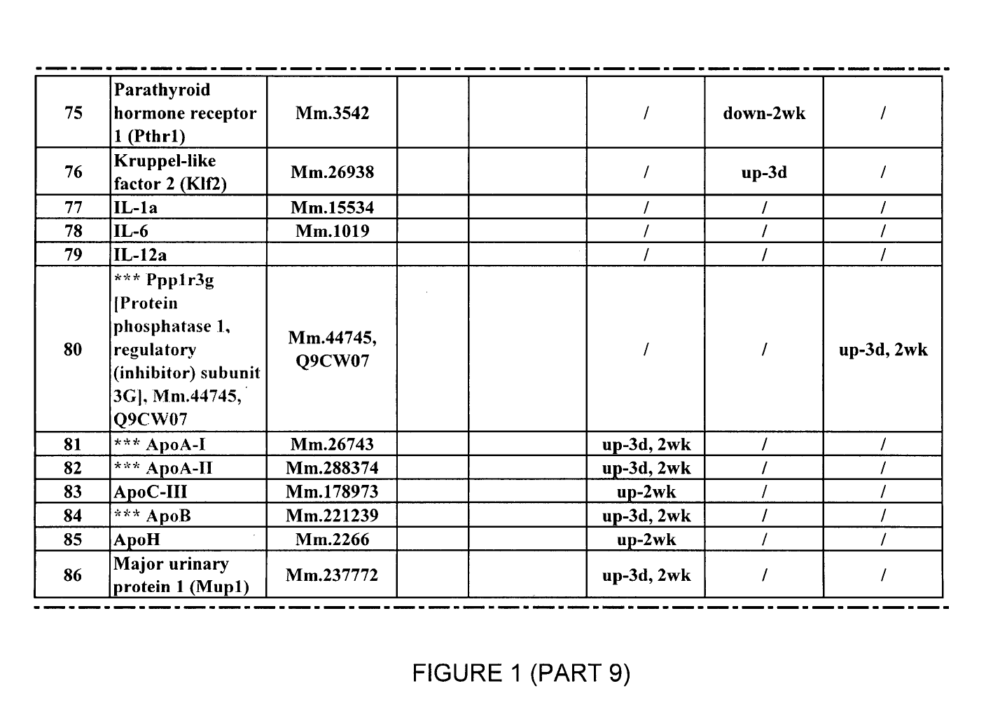

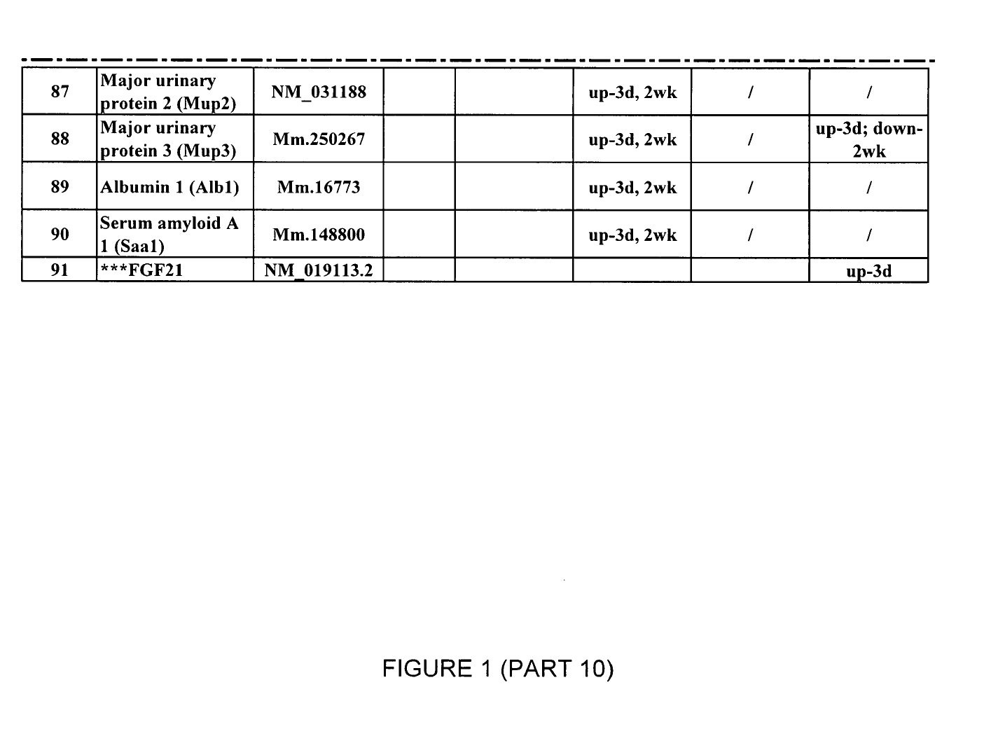

A sirtuin biomarker is a gene having an expression level that is dependent on the level of sirtuin activity and therefore can serve as an indicator of sirtuin activity. The expression level of the sirtuin biomarker may be mRNA expression level and/or protein expression level. Exemplary sirtuin biomarkers are provided herein in Table 1 (FIG. 1).

In certain embodiments, the methods described herein involve detection of the expression level of one or more of the sirtuin biomarkers shown in Table 1 (FIG. 1) in a biological sample. In an exemplary embodiment, the methods described herein may involve detection of the expression level of one or more of the following sirtuin biomarkers: MCP-1, BMP Receptor 1A, Smpd13a, CD14, ApoE, FAS, Transthyretin, FABP1 (liver), Acyl-CoA thioesterase 1, Acyl-CoA thioesterase 2, Aquaporin 4, Rrad, CXCL9, CCL8, Ppp1r3g, ApoA-I, ApoA-II, ApoB, or FGF21. In certain embodiments, the methods described herein involve detection of the expression level of MCP-1. In certain embodiments, the methods described herein involve detection of the expression level of FGF21.

In certain embodiments, methods for identifying individuals that would benefit from treatment with a sirtuin modulator are provided. The methods may involve, for example, determining the expression level of at least one sirtuin biomarker in a biological sample from said subject as compared to a control thereby identifying subjects that would benefit from treatment with a sirtuin modulating compound. Exemplary sirtuin biomarkers are shown in Table 1 (FIG. 1) along with the type of change in expression observed upon treatment with a sirtuin activating compound. For example, MCP-1 expression is decreased upon treatment with a sirtuin activating compound representing an increase in sirtuin activity. Therefore, subjects having an increased MCP-1 expression level as compared to a control may be subjects having a lower than normal level of sirtuin activity that would benefit from treatment with a sirtuin activating compound. Similarly, subjects with a decreased MCP-1 expression level as compared to a control may be subjects having a higher than normal level of sirtuin activity that would benefit from treatment with a sirtuin inhibiting compound. Additionally, a normal level of MCP-1 expression level may also be indicative of subjects that would benefit from treatment with a sirtuin modulating compound. In particular, sirtuin modulation has been shown to be beneficial for treating a variety of diseases and disorders as described further herein. Accordingly, subjects having, for example, a normal level of sirtuin activity may still benefit from an increase or decrease in sirtuin activity.

The level of sirtuin activity based on a determination of the expression level of one or more sirtuin biomarkers may optionally be combined with one or more other indications for a sirtuin mediated disease or disorder in order to identify a subject that would benefit from treatment with a sirtuin modulating compound. For example, a subject having normal to high levels of MCP-1 expression (e.g., normal to low levels of sirtuin activity) and who is overweight or has impaired glucose tolerance may be indicative of a subject who would benefit from treatment with a sirtuin activating compound. Similarly, a subject having normal to low levels of MCP-1 expression (e.g., normal to high levels of sirtuin activity) and who is underweight or anorexic may be indicative of a subject who would benefit from treatment with a sirtuin inhibiting compound.

It should be understood that MCP-1 is merely being used as an example and that the methods described herein are not limited to MCP-1. Rather any of the sirtuin biomarkers provided in Table 1 may be used in a similar manner. For example, Alk3 is upregulated in human white blood cells upon treatment with a sirtuin activator. Therefore, human subjects having normal to low levels of Alk3 represent subjects that could benefit from treatment with a sirtuin activating compound while subjects with normal to high levels of Alk3 represent subjects that could benefit from treatment with a sirtuin inhibiting compound.

In certain embodiments, methods for identifying individuals that are suffering from or at risk for developing a sirtuin mediated disease or disorder are provided. For example, the methods may involve determining the expression level of one or more of the sirtuin biomarkers shown in Table 1 (FIG. 1) as compared to a control thereby identifying a subject suffering from or at risk of developing a sirtuin mediated disease or disorder. Similar to the methods described above, a subject having an increased or decreased expression level of a sirtuin biomarker is indicative of a subject having an altered level of sirtuin activity and therefore suffering from or at risk of developing a sirtuin mediated disease or disorder. For example, a subject having an increased level of MCP-1 expression may be indicative of a subject suffering from or at risk for developing a sirtuin mediated disease or disorder associated with a lower than normal level of sirtuin activity, such as, for example, various neurodegenerative diseases or diabetes. Alternatively, a subject having a decreased level of MCP-1 expression is indicative of a subject suffering from a disease or disorder associated with a higher than normal level of sirtuin activity, such as, for example, cancer or low appetite. Such methods may optionally involve consideration of other indicators of a sirtuin mediated disease or disorder such as weight, glucose tolerance, cognitive ability, etc. Identification and prognosis of sirtuin-mediated disease using a sirtuin biomarker can lead to early diagnosis and proper preventive care.

In certain embodiments, the diagnostic and prognostic methods described herein may further comprise administering a therapeutic treatment to a subject. For example, the level of one or more sirtuin biomarkers may be used to identify a subject suffering from or at risk for developing a sirtuin mediated disease or disorder. The information obtained from the sirtuin biomarker(s) may be used to determine whether the subject would benefit from treatment with a sirtuin modulating compound, e.g., a sirtuin activating compound or sirtuin inhibiting compound. A therapeutic regimen with an appropriate sirtuin modulator may then be chosen and administered to the subject using an appropriate dosing schedule. Such sirtuin modulating therapeutic may optionally be administered in combination with another therapeutic agent that treats or alleviates at least one symptom of the disease or disorder that the subject is susceptible to or suffering from. The sirtuin biomarker profile may be used to aid in designing the therapeutic regimen, e.g., the selection of the sirtuin modulator and/or the dosing regime. For example, the number and/or identity of sirtuin biomarker(s) having altered expression levels as compared to a control, the magnitude of change in the expression level(s), etc. may be used to facilitate selection of an appropriate sirtuin modulating compound and/or in determining the dose and frequency of administration of the therapeutic agent. For example, a subject having an MCP-1 expression level is significantly higher than normal may need a higher dose or more frequent administration schedule than an individual having an MCP-1 expression level that is only slightly higher than normal.

In certain embodiments, the methods described herein may involve measuring the expression level of one or more sirtuin biomarkers in order to detect or monitor sirtuin modulation in a subject. A change in the expression level of a sirtuin biomarker (for example, one or more of the sirtuin biomarkers shown in Table 1 (FIG. 1)), as compared to a control, indicates sirtuin modulation in that subject. In certain embodiments, a decrease in expression level of MCP-1, as compared to a control, indicates sirtuin activation.

In another embodiment, measuring the expression level of a sirtuin biomarker may be useful for monitoring therapeutic treatment with a sirtuin modulating compound. For example, during the course of treatment with a sirtuin therapeutic, the expression level of one or more sirtuin biomarkers from a biological sample of an individual may be determined at one or more time points. A change in the expression level of the sirtuin biomarker, upon treatment with the sirtuin modulator, indicates that the individual is responsive to the treatment. In an exemplary embodiment, administration of a sirtuin activator may result in a decrease in the expression level of MCP-1.

Monitoring of sirtuin activity during the course of treatment with a sirtuin biomarker may also be useful for adjusting the dose or administration schedule of the therapeutic compound. In certain embodiments, a subject is administered a sirtuin therapeutic over time, for example, at least once a day, once a week, once a month, etc. for at least a week, two weeks, one month, two months, six months, one year, or chronically. Expression levels of one or more sirtuin biomarkers may be monitored on a regular or sporadic basis during the course of treatment, for example, sirtuin biomarker expression levels may be measured on a daily, weekly, biweekly, monthly, or bimonthly basis, or once every six months, or once a year. The frequency of sirtuin biomarker expression level monitoring may differ over time, for example, after an optimal treatment regimen (including dosage and/or frequency of administration) is determined, the frequency of monitoring may decrease. In an exemplary embodiment, the methods described herein may involve monitoring the expression level of one or more sirtuin biomarkers at least once a day or at least once a week until an optimized dosage regime is determined. Subsequently, monitoring of the expression level of one or more sirtuin biomarkers is reduce to no more than once per week or no more than once per month.

In certain embodiments, the subject being treated with a sirtuin modulating compound may be suffering from one or more of a variety of sirtuin mediated diseases or disorders. For example, subjects being treated with a sirtuin activating compound may be suffering from a disease or disorder that would benefit from an increase in the level of sirtuin activity, such as, for example, diseases or disorders related to aging or stress, diabetes, obesity, a neurodegenerative disease, chemotherapeutic induced neuropathy, neuropathy associated with an ischemic event, an ocular disease or disorder, cardiovascular disease, a blood clotting disorder, inflammation, or flushing. Subjects being treated with a sirtuin inhibiting compound may be suffering from a disease or disorder that would benefit from a decrease in sirtuin activity, such as, for example, cancer or individuals in need of appetite stimulation or weight gain.

Expression levels of sirtuin biomarker may be determined in a biological sample from a subject. Exemplary biological samples include samples comprising blood, urine, serum, saliva, cells, tissue, and/or hair. Samples may be obtained from a subject using standard techniques. Preferably, biological samples are obtained using minimally invasive, non-surgical procedures, such as, a needle biopsy for obtaining a tissue sample, etc. In various embodiments, biological samples may be taken from healthy individuals, individuals suffering from a disease or disorder that would benefit from sirtuin modulation, or subjects being treated with a sirtuin modulating compound, etc. In certain embodiments, the subject may be a mammal, including, for example, a human. In other embodiments, the subject may be an animal model, including, for example, an animal model of aging, stress, diabetes, obesity, a neurodegenerative disease, chemotherapeutic induced neuropathy, neuropathy associated with an ischemic event, an ocular disease or disorder, cardiovascular disease, a blood clotting disorder, inflammation, flushing, or cancer, or animal model for studying weight gain or appetite stimulation. Suitable animals models are described herein or are known in the art.

In certain embodiments, it may be useful to compare the expression level of one or more sirtuin biomarkers in a biological sample from a subject to a control. The control may be a measure of the expression level of one or more sirtuin biomarkers in a quantitative form (e.g., a number, ratio, percentage, graph, etc.) or a qualitative form (e.g., band intensity on a gel or blot, etc.). A variety of controls may be used. For example, the expression level of one or more sirtuin biomarkers from an individual not being treated with a sirtuin modulator may be used. Expression levels of one or more sirtuin biomarkers from a healthy individual may also be used as a control, e.g., an individual not suffering from a disease or disorder that is present in the individual being treated with a sirtuin modulating compound. Alternatively, the control may be expression levels of one or more sirtuin biomarkers from the individual being treated at a time prior to treatment with the sirtuin modulator or at a time period earlier during the course of treatment with the sirtuin modulator. Still other controls may include expression levels present in a database (e.g., a table, electronic database, spreadsheet, etc.).

### 3. Determination of Sirtuin Biomarker Expression Levels

The expression level of a sirtuin biomarker can be measured by the biomarker's mRNA level, protein level, activity level, or other quantity reflected in or derivable from the biomarker's gene or protein expression data. The expression products of each of the sirtuin biomarkers include both RNA and protein. RNA products of the sirtuin biomarkers are transcriptional products of the sirtuin biomarkers and include populations of hnRNA, mRNA, and one or more spliced variants of mRNA. Protein products of the sirtuin biomarkers may also be measured in accordance with the methods described herein. The protein products of the sirtuin biomarkers include, for example, proteins, protein variants arising from spliced mRNA variants, and post translationally modified proteins.

Any suitable means of measuring the expression of the RNA products of the sirtuin biomarkers can be used in accordance with the methods described herein. For example, the methods may utilize a variety of polynucleotides that specifically hybridize to one or more of the RNA products of the sirtuin biomarkers including, for example, oligonucleotides, cDNA, DNA, RNA, PCR products, synthetic DNA, synthetic RNA, or other combinations of naturally occurring of modified nucleotides which specifically hybridize to one or more of the RNA products of the sirtuin biomarkers. Such polynucleotides may be used in combination with the methods to measure RNA expression described further herein including, for example, array hybridization, RT-PCR, nuclease protection and northern blots.

### Array Hybridization

In one embodiment, the expression level of sirtuin biomarker may be determined suing array hybridization to evaluate the level of RNA expression. Array hybridization utilizes nucleic acid members stably associated with a support that can hybridize with sirtuin biomarker expression products. The length of a nucleic acid member attached to the array can range from 8 to 1000 nucleotides in length and are chosen so as to be specific for the RNA products of the sirtuin biomarkers. The array may comprise, for example, one or more nucleic acid members that are specific for 1, 2, 3, 4, 5, 6, 7, 8, 9, 10, 20, 25, 50, 100, or all of the RNA products of the sirtuin biomarkers shown in Table 1 (FIG. 1), or variants thereof (e.g., splice variants). The nucleic acid members may be RNA or DNA, single or double stranded, and/or may be oligonucleotides or PCR fragments amplified from cDNA. Preferably oligonucleotides are approximately 10-100, 10-50, 20-50, or 20-30 nucleotides in length. Portions of the expressed regions of the sirtuin biomarkers can be utilized as probes on the array. More particularly oligonucleotides complementary to the sirtuin biomarkers genes and or cDNAs derived from the sirtuin biomarker genes are useful. For oligonucleotide based arrays, the selection of oligonucleotides corresponding to the gene of interest which are useful as probes is well understood in the art. More particularly it is important to choose regions which will permit hybridization to the target nucleic acids. Factors such as the Tm of the oligonucleotide, the percent GC content, the degree of secondary structure and the length of nucleic acid are important factors. See for example U.S. Pat. No. 6,551,784.

Arrays may be constructed, custom ordered, or purchased from a commercial vendor. Various methods for constructing arrays are well known in the art. For example, methods and techniques applicable to oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

In an exemplary embodiment, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other methods for constructing arrays include, for example, light-directed methods utilizing masks (e.g., VLSIPS™ methods described, for example, in U.S. Pat. Nos. 5,143,854, 5,510,270 and 5,527,681), flow channel methods (see e.g., U.S. Pat. No. 5,384,261), spotting methods (see e.g., U.S. Pat. No. 5,807,522), pin-based methods (see e.g., U.S. Pat. No. 5,288,514), and methods utilizing multiple supports (see e.g., U.S. Pat. Nos. 5,770,358, 5,639,603, and 5,541,061).

In certain embodiments, an array of nucleic acid members stably associated with the surface of a support is contacted with a sample comprising target nucleic acids under hybridization conditions sufficient to produce a hybridization pattern of complementary nucleic acid members/target complexes in which one or more complementary nucleic acid members at unique positions on the array specifically hybridize to target nucleic acids. The identity of target nucleic acids which hybridize can be determined with reference to location of nucleic acid members on the array.

Control nucleic acid members may be present on the array including nucleic acid members comprising oligonucleotides or nucleic acids corresponding to genomic DNA, housekeeping genes, vector sequences, negative and positive control genes, and the like. Control nucleic acid members are calibrating or control genes whose function is not to tell whether a particular gene of interest is expressed, but rather to provide other useful information, such as background or basal level of expression.

Other control nucleic acids on the array may be used as target expression control nucleic acids and mismatch control nucleotides to monitor non-specific binding or cross-hybridization to a nucleic acid in the sample other than the target to which the probe is directed. Mismatch probes thus indicate whether a hybridization is specific or not. For example, if the target is present, the perfectly matched probes should be consistently brighter than the mismatched probes. In addition, if all control mismatches are present, the mismatch probes are used to detect a mutation.

An array provided herein may comprise a substrate sufficient to provide physical support and structure to the associated nucleic acids present thereon under the assay conditions in which the array is employed, particularly under high throughput handling conditions.

The substrate may be biological, non-biological, organic, inorganic, or a combination of any of these, existing as particles, strands, precipitates, gels, sheets, tubing, spheres, beads, containers, capillaries, pads, slices, films, plates, slides, chips, etc. The substrate may have any convenient shape, such as a disc, square, sphere, circle, etc. The substrate is preferably flat or planar but may take on a variety of alternative surface configurations. The substrate may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art in view of this disclosure.

In certain embodiments, a target nucleic acid sample may comprise total mRNA or a nucleic acid sample corresponding to mRNA (e.g., cDNA) isolated from a biological sample. Total mRNA may be isolated from a given sample using, for example, an acid guanidinium-phenol-chloroform extraction method and polyA+mRNA may be isolated using oligo dT column chromatography or using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987). In certain embodiments, total RNA may be extracted using TRIzol™ reagent (GIBCO/BRL, Invitrogen Life Technologies, Cat. No. 15596). Purity and integrity of RNA may be assessed by absorbance at 260/280 nm and agarose gel electrophoresis followed by inspection under ultraviolet light.

In certain embodiments, it may be desirable to amplify the target nucleic acid sample prior to hybridization. One of skill in the art will appreciate that whatever amplification method is used, if a quantitative result is desired, care must be taken to use a method that maintains or controls for the relative frequencies of the amplified nucleic acids. Methods of quantitative amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid. Detailed protocols for quantitative PCR are provided in PCR Protocols, A Guide to Methods and Applications, Innis et al., Academic Press, Inc. N.Y., (1990).

In certain embodiments, the target nucleic acid sample mRNA is reverse transcribed with a reverse transcriptase and a primer consisting of oligo dT and a sequence encoding the phage T7 promoter to provide single-stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro transcription are well known to those of skill in the art (see, e.g., Sambrook, supra.) and this particular method is described in detail by Van Gelder, et al., 1990, Proc. Natl. Acad. Sci. USA, 87: 1663-1667 who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material thereby permitting expression monitoring even where biological samples are limited.

Detectable labels suitable for use in accordance with the methods described herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The labels may be incorporated by any of a number of means well known to those of skill in the art. For example, the label may be simultaneously incorporated during the amplification step in the preparation of the sample nucleic acids. Thus, for example, polymerase chain reaction (PCR) with labeled primers or labeled nucleotides will provide a labeled amplification product. Additionally, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

In certain embodiments, the fluorescent modifications are by cyanine dyes e.g. Cy-3/Cy-5 dUTP, Cy-3/Cy-5 dCTP (Amersham Pharmacia) or alexa dyes (Khan, et al., 1998, Cancer Res. 58:5009-5013).

In certain embodiments, it may be desirable to simultaneously hybridize two target nucleic acid samples to the array, including, for example, a target nucleic acid sample from a subject (e.g., a subject being treated with a sirtuin modulating compound, or a subject suspected of being at risk or suffering from a sirtuin mediated disease or disorder, etc.) and a control nucleic acid sample (e.g., a nucleic acid sample from a subject not being treated with a sirtuin modulating compound or a healthy individual, etc.). The two target samples used for comparison are labeled with different fluorescent dyes which produce distinguishable detection signals, for example, targets from a control sample are labeled with Cy5 and targets from a subject to be monitored or diagnosed are labeled with Cy3. The differently labeled target samples are hybridized to the same microarray simultaneously. The labeled targets may be purified using methods known in the art, e.g., by ethanol purification or column purification.

In certain embodiments, the target nucleic acid samples will include one or more control molecules which hybridize to control probes on the microarray to normalize signals generated from the microarray. Labeled normalization targets may be, for example, nucleic acid sequences that are perfectly complementary to control oligonucleotides that are spotted onto the microarray as described above. The signals obtained from the normalization controls after hybridization provide a control for variations in hybridization conditions, label intensity, reading efficiency and other factors that may cause the signal of a perfect hybridization to vary between arrays. Signals (e.g., fluorescence intensity) read from all other probes in the array may be divided by the signal (e.g., fluorescence intensity) from the control probes, thereby normalizing the measurements.

Normalization targets may be selected to reflect the average length of the other targets present in the sample or they may be selected to cover a range of lengths. The normalization control(s) also can be selected to reflect the (average) base composition of the other probes in the array. In certain embodiments, only one or a few normalization probes are used and they are selected such that they hybridize well (i.e., have no secondary structure and do not self hybridize) and do not match any target molecules. Normalization probes may be localized at any position in the array or at multiple positions throughout the array to control for spatial variation in hybridization efficiency. For example, normalization controls may be located at the corners or edges of the array as well as in the middle.

Nucleic acid hybridization to an array involves incubating a denatured probe or target nucleic acid member on an array and a target nucleic acid sample under conditions wherein the probe or target nucleic acid member and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches. Methods of optimizing hybridization conditions are well known to those of skill in the art (see, e.g., Laboratory Techniques in Biochemistry and Molecular Biology, Vol. 24: Hybridization With Nucleic acid Probes, P. Tijssen, ed. Elsevier, N.Y., (1993)).

Following hybridization, non-hybridized labeled or unlabeled nucleic acids are removed from the support surface by washing thereby generating a pattern of hybridized target nucleic acid on the substrate surface. A variety of wash solutions are known to those of skill in the art and may be used. The resultant hybridization patterns of labeled, hybridized oligonucleotides and/or nucleic acids may be visualized or detected in a variety of ways, with the particular manner of detection being chosen based on the particular label of the target nucleic acid sample, where representative detection means include scintillation counting, autoradiography, fluorescence measurement, calorimetric measurement, light emission measurement and the like.

Following hybridization, washing step and/or subsequent treatments, the resultant hybridization pattern is detected. In detecting or visualizing the hybridization pattern, the intensity or signal value of the label will be not only be detected but quantified, e.g., the signal from each spot on the hybridized array will be measured and compared to a unit value corresponding to the signal emitted by a known number of end labeled target nucleic acids to obtain a count or absolute value of the copy number of each end-labeled target that is hybridized to a particular spot on the array in the hybridization pattern.

Methods for analyzing the data collected from array hybridizations are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the test nucleic acids from the remaining data. The resulting data is displayed as an image with the intensity in each region varying according to the binding affinity between associated oligonucleotides and/or nucleic acids and the test nucleic acids.

### RT-PCR

In certain embodiments, the level of the expression of the RNA products of the sirtuin biomarkers can be measured by amplifying the RNA products of the biomarkers from a sample using reverse transcription (RT) in combination with the polymerase chain reaction (PCR). In certain embodiments, the RT can be quantitative as would be understood to a person skilled in the art.

Total RNA, or mRNA from a sample may be used as a template and a primer specific to the transcribed portion of a sirtuin biomarkers is used to initiate reverse transcription. Methods of reverse transcribing RNA into cDNA are well known and are described, for example, in Sambrook et al., 1989, supra. Primer design can be accomplished utilizing commercially available software (e.g., Primer Designer 1.0, Scientific Software etc.) or methods that are standard and well known in the art. Primer Software programs can be used to aid in the design and selection of primers include, for example, The Primer Quest software which is available through the following web site link: biotools.idtdna.com/primerquest/. Additionally, the following website links are useful when searching and updating sequence information from the Human Genome Database for use in biomarker primer design:

1) the NCBI LocusLink Homepage: world wide web at ncbi.nlm.nih.gov/LocusLink/, and 2) Ensemble Human Genome Browser: world wide web at ensembl.org/Homo_sapiens, preferably using pertinent biomarker information such as Gene or Sequence Description, Accession or Sequence ID, Gene Symbol, RefSeq #, and/or UniGene #.

General guidelines for designing primers that may be used in accordance with the methods described herein include the following: the product or amplicon length may be ˜100-150 bases, the optimum Tm may be ˜60° C., or about 58-62° C., and the GC content may be ˜50%, or about 45-55%. Additionally, it may be desirable to avoid certain sequences such as one or more of the following: (i) strings of three or more bases at the 3′-end of each primer that are complementary to another part of the same primer or to another primer in order to reduce primer-dimer formation, (ii) sequences within a primer that are complementary to another primer sequence, (iii) runs of 3 or more G's or C's at the 3′-end, (iv) single base repeats greater than 3 bases, (v) unbalanced distributions of G/C- and A/T rich domains, and/or (vi) a T at the 3′-end.

The product of the reverse transcription is subsequently used as a template for PCR. PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts. The method of PCR is well known in the art. PCR, is performed as described in Mullis and Faloona, 1987, Methods Enzymol., 155: 335.

QRT-PCR, which is quantitative in nature, can also be performed to provide a quantitative measure of sirtuin biomarker gene expression levels. In QRT-PCR reverse transcription and PCR can be performed in two steps, or reverse transcription combined with PCR can be performed concurrently. One of these techniques, for which there are commercially available kits such as Taqman (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman system has the additional advantage of not requiring gel electrophoresis and allows for quantification when used with a standard curve.

A second technique useful for detecting PCR products quantitatively is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces a fluorescence proportional to the amount of PCR product. Additionally, other systems to quantitatively measure mRNA expression products are known including Molecular Beacons™.

Additional techniques to quantitatively measure RNA expression include, but are not limited to, polymerase chain reaction, ligase chain reaction, Qbeta replicase (see, e.g., International Application No. PCT/US87/00880), isothermal amplification method (see, e.g., Walker et al. (1992) PNAS 89:382-396), strand displacement amplification (SDA), repair chain reaction, Asymmetric Quantitative PCR (see, e.g., U.S. Publication No. US200330134307A1) and the multiplex microsphere bead assay described in Fuja et al., 2004, Journal of Biotechnology 108:193-205.

The level of gene expression can be measured by amplifying RNA from a sample using transcription based amplification systems (TAS), including nucleic acid sequence amplification (NASBA) and 3SR. See, e.g., Kwoh et al (1989) PNAS USA 86:1173; International Publication No. WO 88/10315; and U.S. Pat. No. 6,329,179. In NASBA, the nucleic acids may be prepared for amplification using conventional phenol/chloroform extraction, heat denaturation, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once with a polymerase such as T7 or SP6. The resulting products, whether truncated or complete, indicate target specific sequences.

Several techniques may be used to separate amplification products. For example, amplification products may be separated by agarose, agarose-acrylamide or polyacrylamide gel electrophoresis using conventional methods. See Sambrook et al., 1989. Several techniques for detecting PCR products quantitatively without electrophoresis may also be used (see for example PCR Protocols, A Guide to Methods and Applications, Innis et al., Academic Press, Inc. N.Y., (1990)). For example, chromatographic techniques may be employed to effect separation. There are many kinds of chromatography which may be used: adsorption, partition, ion-exchange and molecular sieve, HPLC, and many specialized techniques for using them including column, paper, thin-layer and gas chromatography (Freifelder, Physical Biochemistry Applications to Biochemistry and Molecular Biology, 2nd ed., Wm. Freeman and Co., New York, N.Y., 1982).

Amplification products must be visualized in order to confirm amplification of the nucleic acid sequences of interest. One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

Alternatively, visualization may be achieved indirectly. Following separation of amplification products, a labeled, nucleic acid probe is brought into contact with the amplified nucleic acid sequence of interest. The probe may be conjugated to a chromophore, radiolabeled, or conjugated to a binding partner, such as an antibody or biotin, where the other member of the binding pair carries a detectable moiety.

Additionally, detection may be carried our using Southern blotting and hybridization with a labeled probe. The techniques involved in Southern blotting are well known to those of skill in the art and may be found in many standard books on molecular protocols. See Sambrook et al., 1989, supra. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

### Nuclease Protection Assays

In certain embodiments, Nuclease protection assays (including both ribonuclease protection assays and S1 nuclease assays) can be used to detect and quantitate RNA products of the sirtuin biomarkers. In nuclease protection assays, an antisense probe (e.g., radiolabeled or nonisotopic labeled) hybridizes in solution to an RNA sample. Following hybridization, single-stranded, unhybridized probe and RNA are degraded by nucleases. An acrylamide gel is used to separate the remaining protected fragments. Typically, solution hybridization can accommodate up to ˜100 μg of sample RNA whereas blot hybridizations may only be able to accommodate ˜20-30 μg of RNA sample.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

### Northern Blots

A standard Northern blot assay can also be used to ascertain an RNA transcript size, identify alternatively spliced RNA transcripts, and the relative amounts of RNA products of the sirtuin biomarkers, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabeled cDNA, either containing the full-length, single stranded DNA or a fragment of that DNA sequence may be any length up to at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Non-limiting examples of isotopes include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme may be conjugated to the selected probe by reaction with bridging molecules such as carbodiimides, diisocyanates, glutaraldehyde and the like. Any enzymes known to one of skill in the art can be utilized, including, for example, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

### Protein Products

The expression level of a sirtuin biomarker may also be measured by the biomarker's protein level using any art-known method. Traditional methodologies for protein quantification include 2-D gel electrophoresis, mass spectrometry and antibody binding. Preferred method for assaying biomarker protein levels in a biological sample include antibody-based techniques, such as immunoblotting (western blotting), immunohistological assay, enzyme linked immunosorbent assay (ELISA), radioimmunoassay (RIA), or protein chips. For example, a biomarker-specific monoclonal antibodies can be used both as an immunoadsorbent and as an enzyme-labeled probe to detect and quantify the biomarker. The amount of biomarker present in the sample can be calculated by reference to the amount present in a standard preparation using a linear regression computer algorithm. In another embodiment, sirtuin biomarkers may be immunoprecipitated from a biological sample (e.g., directly from urine or serum or from a lysate of cells, etc.) using an antibody specific for the biomarker. The isolated proteins may then be run on an SDS-PAGE gel and blotted (e.g., to nitrocellulose or other suitable material) using standard procedures. The blot may then be probed with an anti-biomarker specific antibody to determine the expression level of the sirtuin biomarkers.

Gel electrophoresis, immunoprecipitation and mass spectrometry may be carried out using standard techniques, for example, such as those described in Molecular Cloning A Laboratory Manual, 2^{nd} Ed., ed. by Sambrook, Fritsch and Maniatis (Cold Spring Harbor Laboratory Press: 1989), Harlow and Lane, Antibodies: A Laboratory Manual (1988 Cold Spring Harbor Laboratory), G. Suizdak, Mass Spectrometry for Biotechnology (Academic Press 1996), as well as other references cited herein.

As used herein, the term “antibody” (Ab) or “monoclonal antibody” (mAb) is meant to include intact molecules as well as antibody portions (such as, for example, Fab, Fab′, F(ab′)₂, Fv, single chain Fv, or Fd) which are capable of specifically binding to a sirtuin biomarker.

Antibodies suitable for isolation and detection of sirtuin biomarkers, e.g., the biomarkers shown in Table 1, may be purchased commercially from a variety of sources. For example, antibodies specific for human MCP-1 may be purchased from Abcam Inc., Cambridge, Mass., or BioLegend San Diego, Calif. 92121. Antibodies specific for sirtuin biomarkers may also be produced using standard techniques. Generally applicable methods for producing antibodies are well known in the art and are described extensively in references cited herein, e.g., Current Protocols in Immunology and Using Antibodies: A Laboratory Manual. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. In general, preferred antibodies will possess high affinity, e.g., a K_{d} of <200 nM, and preferably, of <100 nM for a specific sirtuin biomarker.

The expression level of a sirtuin biomarker can be measured by the biomarker's activity level using any art-known method. For example, one exemplary sirtuin biomarker, MCP-1, is a potent chemoattractant for monocytes in vitro and in vivo. In one study, MCP-1 activity was determined based on the retinal detachment (RD)-induced photoreceptor apoptosis rate, as quantified by TUNEL (Nakazawa, T., et al., Proc. Natl. Acad. Sci. USA 2007, 104, 2425-2430). Further information about the activity of certain exemplary sirtuin biomarkers is provided below.

Monocyte chemotactic protein-1 (MCP-1). MCP-1 is a member of the CC chemokine family and a potent chemoattractant for monocytes in vitro and in vivo. Much evidence exists supporting a key role for MCP-1 in the pathogenesis of atherosclerosis. There is also growing evidence that MCP-1 may play an important pathogenic role in other cardiovascular diseases such as myocardial ischemia and congestive heart failure. Recently, increased expression of MCP-1 was reported in vitreous humor samples of patients with retinal detachment and several other visual disorders. Activation of MCP-1 is also linked to retinal detachment-induced photoreceptor apoptosis.

Bone morphogenetic protein receptor, type IA (BMP Receptor 1A). Cellular responses to bone morphogenetic proteins (BMPs) have been shown to be mediated by the formation of hetero-oligomeric complexes of the type I and type II serine/threonine kinase receptors. BMP receptor 1A (BMPR-1A), also known as activin receptor-like kinase (ALK)-3, is a one of seven known type I serine/threonine kinases that are required for the signal transduction of TGF-(3 family cytokines. In contrast to the TGF-β receptor system in which the type I receptor does not bind TGF-β in the absence of the type II receptor, type I receptors involved in BMP signaling (including BMPR-IA, BMPR-IB/ALK-6, and ActR-I/ALK-2) can independently bind the various BMP family proteins in the absence of type II receptors. Recombinant soluble BMPR-IA binds BMP-2 and -4 with high-affinity in solution and is a potent BMP-2/4 antagonist in vitro. BMPR-IA is ubiquitously expressed during embryogenesis. In adult tissues, BMPR-IA mRNA is also widely distributed; with the highest expression levels found in skeletal muscle. The extracellular domain of BMPR-IA shares little amino acid sequence identity with the other mammalian ALK type I receptor kinases, but the cysteine residues are conserved. Human and mouse BMPR-IA are highly conserved and share 98% sequence identity.

Sphingomyelin Phosphodiesterase, acid-like 3A (Smpd13a). Smpd13a is a phosphodiesterase enzyme which acts upon sphingomyelin. A deficiency in this enzyme is associated with Niemann-Pick disease. Smpd13a protein is found to be differentially expressed in 0.8 of 12 bladder tumors relative to corresponding normal urothelial tissue. Transient transfection of bladder tumor cell lines showed that Deleted in Bladder Cancer 1 (DBCCR1) over-expression in human bladder tumor cells results in the up-regulation of Smpd13a RNA and protein expression.

CD14 Antigen. CD14 is a membrane-associated glycosylphosphatidylinositol-linked protein expressed at the surface of cells, especially macrophages. CD14 takes its name from it inclusion in the cluster of differentiation group of cell surface marker proteins. CD14 acts as a co-receptor (along with the Toll-like receptor TLR 4 and MD-2) for the detection of bacterial lipopolysaccharide. CD14 was the first described pattern recognition receptor. A soluble form sCD14 is secreted by the liver and monocytes and is sufficient in low concentrations to confer LPS-responsiveness to cells which otherwise do not express CD14.

Apolipoprotein E (ApoE). ApoE, a main apoprotein of the chylomicron, binds to a specific receptor on liver cells and peripheral cells. ApoE is essential for the normal catabolism of triglyceride-rich lipoprotein constituents. ApoE was initially recognized for its importance in lipoprotein metabolism and cardiovascular disease. More recently, it has been studied for its role in several biological processes not directly related to lipoprotein transport, including Alzheimer's disease, immunoregulation, and cognition. Defects in ApoE result in familial dysbetalipoproteinemia, or type III hyperlipoproteinemia (HLP III), in which increased plasma cholesterol and triglycerides are the consequence of impaired clearance of chylomicron, VLDL and LDL remnants. The ApoE protein is 299 amino acids long and transports lipoproteins, fat-soluble vitamins, and cholesterol into the lymph system and then into the blood. It is synthesized principally in the liver, but has also been found in other tissues such as the brain, kidneys, and spleen. In the nervous system, non-neuronal cell types, most notably astroglia and microglia, are the primary producers of ApoE, while neurons preferentially express the receptors for ApoE.

Fatty Acid Synthetase (FAS). FAS, is the sole enzyme capable of the reductive de novo synthesis of long-chain fatty acids from acetyl-CoA, malonyl-CoA, and nicotinamide adenine dinucleotide phosphate or NADPH. Whereas FAS catalyzes the synthesis of long-chain fatty acids, the breakdown of fatty acids by beta-oxidation is regulated by carnitine palmitoyltransferase-1, the rate-limiting enzyme for the entry of fatty acids into the mitochondria for oxidation. Two transcription factors, Upstream Stimulatory Factor (USF) and Sterol Regulatory Element Binding Protein-1c (SREBP-1c), seem to play a dominant and possibly cooperative role in regulating FAS transcription. Inhibition of FAS using cerulenin or synthetic FAS inhibitors such as C75 reduces food intake and induces profound reversible weight loss. Subsequent studies reveal that C75 also stimulates CPT-1 and increases beta-oxidation. Hypotheses as to the mechanisms by which C75 and cerulenin mediate their effects have been proposed. Centrally, these compounds alter the expression profiles of feeding-related neuropeptides, often inhibiting the expression of orexigenic peptides. Whether through centrally mediated or peripheral mechanisms, C75 also increases energy consumption, which contributes to weight loss. In vitro and in vivo studies demonstrate that at least part of C75's effects is mediated by modulation of AMP-activated protein kinase (AMPK), a known peripheral energy-sensing kinase. Collectively, these data suggest a role for fatty acid metabolism in the perception and regulation of energy balance. In addition, FAS is extremely low in nearly all nonmalignant adult tissues, whereas it is significantly up-regulated or activated in many cancer types, making it an interesting target for cancer therapy.

Transthyretin (TTR). Transthyretin is a serum and cerebrospinal fluid carrier of the thyroid hormone thyroxine (T4). It functions in concert with two other thyroid hormone binding proteins, thyroxine-binding globulin (TBG) and albumin. Transthyretin is a 55 kDa homotetramer with a dimer of dimers configuration that is synthesized in the liver, choroid plexus and retinal pigment epithelium. Each monomer is a 127 residue polypeptide rich in β-sheet structure. Association of two monomers forms an extended β-sandwich. Further association of another identical set of monomers produces the homotetrameric structure. The two thyroxine binding sites per tetramer sit at the interface between the latter set of dimers. Transthyretin is known to be associated with the amyloid diseases senile systemic amyloidosis (SSA), familial amyloid polyneuropathy (FAP), and familial amyloid cardiomyopathy (FAC). Numerous other small molecules are known to bind in the thyroxine binding sites, including many natural products (such as resveratrol), drugs (diflunisal, flufenamic acid), and toxins PCB.

Fatty acid binding protein 1, liver (FABP1). Liver fatty-acid-binding protein (FABP1) is found in high abundance in the hepatocyte cytosol, but associates also in the hepatocyte nucleus in a specific ligand-dependent manner. It facilitates the cellular uptake, transport and metabolism of fatty acids and is involved in the regulation of gene expressions and cell differentiation. FABP1 belongs to the family of intracellular lipid binding proteins, having a 10-stranded β-clam structure confining a lipid-binding cavity gated by two short anti-parallel helices; however, FABP1 is unique in this family in that the ligand pocket is unusually large and therefore capable of binding two molar equivalents of long-chain fatty acids and also larger ligands such as heme. FABP1 binding and transport of peroxisome proliferators, especially leukotriene D4 antagonists, are implicated in side-effects of anti-inflammatory asthma therapy.

Acyl-CoA thioesterase 1 (Acot1) and Acyl-CoA thioesterase 2 (Acot2). The maintenance of cellular levels of free fatty acids and acyl-CoAs, the activated form of free fatty acids, is extremely important, as imbalances in lipid metabolism have serious consequences for human health. Acyl-coenzyme A (CoA) thioesterases (Acots) hydrolyze acyl-CoAs to the free fatty acid and CoASH, and thereby have the potential to regulate intracellular levels of these compounds. Both mouse and human Acot gene clusters have been characterized, each comprising gene duplications encoding ACOT1 (in cytosol), ACOT2 (in mitochondria), and ACOT3-6 (in peroxisomes).

Aquaporin 4. Aquaporins are a class of integral membrane proteins or more commonly referred to as a class of major intrinsic proteins (MIP) that form pores in the membrane of biological cells. Aquaporins selectively conduct water molecules in and out, while preventing the passage of ions and other solutes. Aquaporins are commonly composed of four (typically) identical subunit proteins in mammals, with each monomer acting as a water channel. Genetic defects involving aquaporin genes have been associated with several human diseases. Aquaporin 1 is a widely expressed water channel. Aquaporin 2 is found in the apical cell membranes of the kidney's collecting duct principal cells and in intracellular vesicles located throughout the cell. Aquaporins 3 and 4 are found in the basolateral cell membrane of principal collecting duct cells and provide a pathway for water to exit these cells. In kidney, Aquaporin 4 is constitutively expressed. Aquaporin 4 is expressed in astrocytes and are upregulated by direct insult to the central nervous system. Aquaporin 7 was reported to be a glycerol channel expressed in adipocytes and playing a role in lipolysis.

Ras-Related Associated with Diabetes (Rrad). Rrad is a 29-kD protein and a member of the Ras-guanosine triphosphatase superfamily. Messenger RNA of Rrad is expressed primarily in skeletal and cardiac muscle and is increased an average of 8.6-fold in the muscle of type II diabetics as compared with normal individuals. Elevated levels of Rrad mRNA in skeletal muscle has been reported by some to be associated with insulin resistance in human diabetic patients.

Chemokine (C—X—C motif) ligand 9 (CXCL9). CXCL9 is a small cytokine belonging to the CXC chemokine family that is also known as Monokine induced by gamma interferon (MIG). CXCL9 is a T-cell chemoattractant, which is induced by IFN-γ. It is closely related to two other CXC chemokines called CXCL10 and CXCL11, whose genes are located near the gene for CXCL9 on human chromosome 4. CXCL9, CXCL10 and CXCL11 all elicit their chemotactic functions by interacting with the chemokine receptor CXCR3.

Chemokine (C—C motif) ligand 8 (CCL8). CCL8 is a small cytokine belonging to the CC chemokine family that was once called monocyte chemotactic protein-2 (MCP-2). The CCL8 protein is produced as a precursor containing 109 amino acids, which is cleaved to produce mature CCL8 containing 75 amino acids. The gene for CCL8 is encoded by 3 exons and is located within a large cluster of CC chemokines on chromosome 17q11.2 in humans. MCP-2 is chemotactic for and activates a many different immune cells, including mast cells, eosinophils and basophils, (that are implicated in allergic responses), and monocytes, T cells, and NK cells that are involved in the inflammatory response. CCL8 elicits its effects by binding to several different cell surface receptors called chemokine receptors. These receptors include CCR1, CCR2B and CCR5.

Protein Phosphatase 1, Regulatory (inhibitor) Subunit 3G (Ppp1r3g). Protein phosphatase 1 (PP1) is a major eukaryotic protein serine/threonine phosphatase that regulates an enormous variety of cellular functions through the interaction of its catalytic subunit (PP1c) with over fifty different established or putative regulatory subunits. Ppp1r3g is a newly identified regulatory subunit that targets the glycogen-binding regions of PP1. It contains the canonical -RVxF- motif that mediates interaction with PP1, as well as putative modules for targeting to glycogen and facilitating interaction with PP1 substrates.

Apolipoproteins (ApoA-I, ApoA-II, and ApoB). Apolipoproteins are lipid-binding proteins which are the constituents of the plasma lipoproteins, sub-microscopic spherical particles that transport dietary lipids through the bloodstream from the intestine to the liver, and endogenously synthesized lipids from the liver to tissues that can store them (adipocytes), metabolize them (muscle, heart, lung), or secrete them (breast). The amphipathic properties of apolipoproteins solubilize the hydrophobic lipid constituents of lipoproteins, but apolipoproteins also serve as enzyme co-factors, receptor ligands, and lipid transfer carriers that regulate the intravascular metabolism of lipoproteins and their ultimate tissue uptake.

There are five major classes of apolipoproteins, and several sub-classes: A (apo A-I, apo A-II, apo A-IV, and apo A-V); B (apo B48 and apo B100), C (apo C-I, apo C-II, apo C-III, and apo C-IV); D, E, H, and J. Hundreds of genetic polymorphisms of the apolipoproteins have been described, and many of them alter their structure and function.

Apolipoprotein synthesis in the intestine is regulated principally by the fat content of the diet. Apolipoprotein synthesis in the liver is controlled by a host of factors, including dietary composition, hormones (insulin, glucagon, thyroxin, estrogens, androgens), alcohol intake, and various drugs (statins, nicotinic acid, and fibric acids).

Fibroblast Growth Factor 21 (FGF-21 or FGF21). The fibroblast growth factor (FGF) proteins belong to a family of signaling molecules that regulate growth and differentiation of a variety of cell types. FGF-21 has been reported to be preferentially expressed in the liver (Nishimura et al., Biochimica et Biophysica Acta, 1492:203-206, 2000; WO01/36640; and WO01/18172). The human FGF-21 gene and the corresponding gene expression products are described in United States Patent Application 20070238657. FGF21 has been described as a treatment for ischemic vascular disease, wound healing, and diseases associated with loss of pulmonary, bronchia or alveolar cell function and numerous other disorders. More recently, FGF-21 has been shown to stimulate glucose-uptake in mouse 3T3-L1 adipocytes after treatment in the presence and absence of insulin, and to decrease fed and fasting blood glucose, triglycerides, and glucagon levels in ob/ob and db/db mice and 8 week old ZDF rats in a dose-dependant manner, thus, providing the basis for the use of FGF-21 as a therapy for treating diabetes and obesity (WO03/011213). Potential other benefits of upregulating FGF21 include reducing the mortality and morbidity in critically ill patients, such as those experiencing an unstable hypermetabolic state arising, for example, from changes in substrate metabolism which may lead to relative deficiencies in some nutrients. Generally, in an unstable metabolic state, there is increased oxidation of both fat and muscle. In addition, critically ill patients could benefit from increased FGF-21 because it reduces the risk of mortality and morbidity, for instance in patients that experience systemic inflammatory response syndrome or respiratory distress.

### 4. Screening Assays

In other aspects, the invention provides methods for identifying compounds that modulate sirtuin activity. The assays may comprise contacting a cell that expresses a sirtuin protein with a test compound and determining the expression level of one or more sirtuin biomarkers (e.g., one or more of the biomarkers shown in Table 1). In certain embodiments, the screening assays described herein may involve determining the expression level of 1, 2, 3, 4, 5, 10, 15, 20, 25, or more, of the sirtuin biomarkers shown in Table 1.

In certain embodiments, the methods described herein involve detection of the expression level of one or more of the sirtuin biomarkers shown in Table 1 (FIG. 1). In an exemplary embodiment, the methods described herein may involve detection of the expression level of one or more of the following sirtuin biomarkers: MCP-1, BMP Receptor 1A, Smpd13a, CD 14, ApoE, FAS, Transthyretin, FABP1 (liver), Acyl-CoA thioesterase 1, Acyl-CoA thioesterase 2, Aquaporin 4, Rrad, CXCL9, CCL8, Ppp1r3g, ApoA-I, ApoA-II, ApoB, or FGF21. In certain embodiments, the methods described herein involve detection of the expression level of MCP-1. In certain embodiments, the methods described herein involve detection of the expression level of FGF21.

Merely as an example, an increase of MCP-1 expression in cell upon contact with a test compound as compared to a control, is indicative of a test compound that activates sirtuin activity. Alternatively, a decrease of MCP-1 expression in a cell upon contact with a test compound as compared to a control, is indicative of a test compound that inhibits sirtuin activity. Similar methods may be conducted using other biomarkers shown in Table 1 (FIG. 1) or combinations thereof. Table 1 shows the effects on biomarker expression in the presence of a sirtuin activating compound and therefore similar effects would be expected in the assays described herein wherein a test compound has sirtuin activating effects. Similarly, the opposite effects on expression would be expected to those shown in Table 1 when a test compound exhibited sirtuin inhibiting effects.

The expression level of a sirtuin biomarker can be measured by the biomarker's mRNA level, protein level, activity level, or other quantity reflected in or derivable from the biomarker's gene or protein expression data. Exemplary methods for determining expression levels of sirtuin biomarkers are provided in the exemplification section herein. Standard methods and compositions for determining the amount of RNA or protein product of a sirtuin biomarker can be utilized. Such methods and compositions are described in detail above.

In certain embodiments, the cell based assays described herein may utilize a cell that endogenously expresses a sirtuin protein. Alternatively, cells may be engineered so as to express a sirtuin (e.g., integration of a sirtuin gene into the genome of the host cell, expression from a plasmid containing a sirtuin sequence, etc.). In certain embodiments, cells useful in the assays described herein endogenously express at least one sirtuin biomarker listed in Table 1 (or a homolog thereof). In other embodiments, cells may be engineered so as to express one or more sirtuin biomarkers listed in Table 1. Cells can be engineered to express a sirtuin, sirtuin biomarker or other sequence using techniques well-known in the art. Examples of such techniques include, but are not to, calcium phosphate precipitation (see, e.g., Graham & Van der Eb, 1978, Virol. 52:546), dextran-mediated transfection, calcium phosphate mediated transfection, polybrene mediated transfection, protoplast fusion, electroporation, encapsulation of the nucleic acid in liposomes, and direct microinjection of the nucleic acid into nuclei.

A sirtuin protein refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Homologs, e.g., orthologs and paralogs, domains, fragments, variants and derivatives of the foregoing may also be used in accordance with the methods described herein.

In an exemplary embodiment, the methods described herein may be used to determine the activity of a SIRT1 protein. A SIRT1 protein refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), and human SIRT2 (GenBank Accession No. NM_{—}012237, NM_{—}030593, NP_{—}036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP_{—}501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. SIRT1 proteins also include homologs (e.g., orthologs and paralogs), variants, or fragments, of GenBank Accession Nos. NP_{—}036370, NP_{—}501912, NP_{—}085096, NP_{—}036369, or P53685.

In one embodiment, the methods described herein may be used to determine the activity of a SIRT3 protein. A SIRT3 protein refers to a member of the sirtuin deacetylase protein family and/or to a homolog of a SIRT1 protein. In one embodiment, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In another embodiment, a SIRT3 protein includes a polypeptide comprising a sequence consisting of, or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. SIRT3 proteins also include homologs (e.g., orthologs and paralogs), variants, or fragments, of GenBank Accession Nos. AAH01042, NP_{—}036371, NP_{—}001017524, or NP_{—}071878.

In another embodiment, a biologically active portion of a sirtuin may be used in accordance with the methods described herein. A biologically active portion of a sirtuin refers to a portion of a sirtuin protein having a biological activity, such as the ability to deacetylate. Biologically active portions of sirtuins may comprise the core domain of a sirtuin. Biologically active portions of SIRT1 having GenBank Accession No. NP_{—}036370 that encompass the NAD+ binding domain and the substrate binding domain, for example, may include without limitation, amino acids 62-293 of GenBank Accession No. NP_{—}036370, which are encoded by nucleotides 237 to 932 of GenBank Accession No. NM_{—}012238. Therefore, this region is sometimes referred to as the core domain. Other biologically active portions of SIRT1, also sometimes referred to as core domains, include about amino acids 261 to 447 of GenBank Accession No. NP_{—}036370, which are encoded by nucleotides 834 to 1394 of GenBank Accession No. NM_{—}012238; about amino acids 242 to 493 of GenBank Accession No. NP_{—}036370, which are encoded by nucleotides 777 to 1532 of GenBank Accession No. NM_{—}012238; or about amino acids 254 to 495 of GenBank Accession No. NP_{—}036370, which are encoded by nucleotides 813 to 1538 of GenBank Accession No. NM_{—}012238. In another embodiment, a biologically active portion of a sirtuin may be a fragment of a SIRT3 protein that is produced by cleavage with a mitochondrial matrix processing peptidase (MPP) and/or a mitochondrial intermediate peptidase (MIP).

Cells useful in accordance with the assays provided herein may be either prokaryotic or eukaryotic. In exemplary embodiments, host cells are cultured mammalian cells, preferably human cells, that endogenously express a sirtuin protein and one or more of the sirtuin biomarkers listed in Table 1. In certain embodiments, the cells may be suspended in culture or may be contained within a non-human animal. For example, assays may be carried out by administering a putative sirtuin modulating compound to a non-human animal, obtaining a biological sample from said animal, and determining the expression level of one or more sirtuin biomarkers in the biological sample. The non-human animal may be, for example, an animal model of a sirtuin mediated disease or disorder or a normal animal. Additionally, assays may be carried out using cells contained in biological samples from a subject such as a mammal, including a human subject. For example, a biological sample may be removed from a subject, treated with a sirtuin modulating compound, and then the expression level of one or more sirtuin biomarker in the sample may be determined. The subject may be, for example, a human subject suffering from a sirtuin mediated disease or disorder or an animal model of a sirtuin mediated disease or disorder.

In other embodiments, any cell-free extract that permits the translation, and optionally the transcription, of a nucleic acid can be used in accordance with the methods described herein. The cell-free extract may be isolated from cells of any origin. For example, the cell-free translation extract may be isolated from human cells, cultured mouse cells, cultured rat cells, Chinese hamster ovary (CHO) cells, Xenopus oocytes, rabbit reticulocytes, wheat germ, or rye embryo (see, e.g., Krieg & Melton, 1984, Nature 308:203 and Dignam et al., 1990 Methods Enzymol. 182:194-203). Alternatively, the cell-free translation extract, e.g., rabbit reticulocyte lysates and wheat germ extract, can be purchased commercially, e.g., from Promega, (Madison, Wis.). In an exemplary embodiment, the cell-free extract is an extract isolated from human cells, such as, for example, HeLa cells or lymphocytes.

In certain embodiments, the methods described herein for identifying sirtuin modulating compounds may utilize a sirtuin activatable cell line. A sirtuin activatable cell line comprises a relatively low endogenous level of one or more sirtuin proteins (e.g., the amount of sirtuin activity in the cell is not saturating and an increase in activity is observable) and a relatively low level of mitochondria and/or oxidative phosphorylation capacity (e.g., the amount of mitochondria and/or oxidative phosphorylation in the cell is not saturating and an increase in ATP levels is observable). Exemplary sirtuin activatable cell lines include, for example, NCI-H358 and MCS7.

In certain embodiments, the screening methods described herein involve comparing the expression level of one or more sirtuin biomarkers in the presence of a test compound to a control. In various embodiments, the control may be a duplicate assay conducted in the absence of a test compound or a duplicate assay conducted in the presence of a test compound having known sirtuin modulating activity (e.g., an activator, inhibitor, or a compound having no sirtuin modulating activity). In yet other embodiments, a control may be a reference number in a database.

In certain embodiments, the screening assays described herein can use reporter gene-based assays to identify sirtuin modulating compounds. For example, a reporter gene under the control of the upstream regulatory sequences of a sirtuin biomarker can be used to determine the effects of a test compound on the expression of the sirtuin biomarker, thereby reflecting the effects of the test compound on sirtuin activity. In particular, a method may comprise, for example, (a) contacting a cell expressing a reporter gene construct comprising a reporter gene operably linked to a regulatory element of a sirtuin biomarkers (e.g., a promoter/enhancer element) with a test compound; (b) measuring the expression of said reporter gene; and (c) comparing the amount in (a) to that present in a corresponding control cell that has not been contacted with the test compound, so that if the amount of expressed reporter gene is altered relative to the amount in the control cell, a compound that modulates sirtuin activity is identified. In another embodiment, methods for identifying a sirtuin modulating may comprise: (a) contacting a cell-free extract and a reporter gene construct comprising a reporter gene operably linked to a regulatory element of a sirtuin biomarkers (e.g., a promoter/enhancer element) with a test compound; (b) measuring the expression of said reporter gene; and (c) comparing the amount in (a) to that present in a corresponding control that has not been contacted with the test compound, so that if the amount of expressed reporter gene is altered relative to the amount in the control, a sirtuin modulating compound is identified.

Any reporter gene well-known to one of skill in the art may be used in accordance with the methods described herein. Reporter genes refer to a nucleotide sequence encoding an RNA transcript or protein that is readily detectable either by its presence (by, e.g., RT-PCR, Northern blot, Western Blot, ELISA, etc.) or activity. Non-limiting examples of reporter genes include, for example, chloramphenicol acetyltransferase (CAT; transfers radioactive acetyl groups to chloramphenicol or detection by thin layer chromatography and autoradiography), beta-galactosidase (GAL; hydrolyzes colorless galactosides to yield colored products), beta-glucuronidase (GUS; hydrolyzes colorless glucuronides to yield colored products), luciferase (LUC; oxidizes luciferin, emitting photons), green fluorescent protein (GFP; fluorescent protein without substrate), secreted alkaline phosphatase (SEAP; luminescence reaction with suitable substrates or with substrates that generate chromophores), horseradish peroxidase (HRP; in the presence of hydrogen oxide, oxidation of 3,3′,5,5′-tetramethylbenzidine to form a colored complex), and alkaline phosphatase (AP; luminescence reaction with suitable substrates or with substrates that generate chromophores). Nucleotide sequences for suitable reporter genes can be obtained, e.g., from the literature or a database such as GenBank. The nucleotide sequence of the reporter gene may be linked to a regulatory sequence for a sirtuin biomarker using methods well-known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY), to generate reporter genes suitable for use in accordance with the methods described herein.

In certain embodiments, the invention provides methods for screening for compounds that modulate expression levels of one or more sirtuin biomarkers. In certain embodiments, the methods described herein may be used to identify a test compound that decreases or increases sirtuin biomarker expression by at least about 2-fold, 3-fold, 5-fold, 10-fold, 15-fold, 20-fold, 25-fold, or more, relative to the biomarker expression level in the absence of the test compound.

Test compounds to be tested for activity in the assays described herein can include proteins (including post-translationally modified proteins), peptides (including chemically or enzymatically modified peptides), or small molecules (including carbohydrates, steroids, lipids, anions or cations, drugs, small organic molecules, oligonucleotides, antibodies, and genes encoding proteins of the agents or antisense molecules), including libraries of compounds. The test compounds can be naturally occurring (e.g., found in nature or isolated from nature) or can be non-naturally occurring (e.g., synthetic, chemically synthesized or man-made).

If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J Med Chem. 37,2678, 1994; Cho et al., Science 261, 1303, 1993; Carell et al., Angew. Chem. Int. Ed Engl. 33, 2059, 1994; Carell et al., Angew. Chem. Int. Ed. Engl. 33, 2061; Gallop et al., J. Med Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

Test compounds can be screened for the ability to modulate sirtuin biomarker expression or sirtuin deacetylase activity using high throughput screening. Using high throughput screening, many discrete compounds can be tested in parallel so that large numbers of test compounds can be quickly screened. The most widely established techniques utilize 96-well microtiter plates. In addition to the plates, many instruments, materials, pipettors, robotics, plate washers, and plate readers are commercially available to fit the 96-well format.

Alternatively, free format assays, or assays that have no physical barrier between samples, can be used. Assays involving free formats are described, for example, in Jayawickreme et al., Proc. Natl. Acad. Sci. U.S.A. 19, 1614-18 (1994); Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995); and Salmon et al., Molecular Diversity 2, 57-63 (1996). Another high throughput screening method is described in Beutel et al., U.S. Pat. No. 5,976,813.

In certain embodiments, the biomarker assays described herein may be used as a primary assay to identify putative sirtuin modulating compounds. Such assays may further comprise additional in vitro assays to directly measure the effects on sirtuin deacetylase activity in the presence of the putative sirtuin modulating compound. Any suitable assay for determining sirtuin deacetylase activity may be used in accordance with the methods described herein. The deacetylase assays may be used to identify compounds that either activate sirtuin deacetylase activity or compounds that inhibit sirtuin deacetylase activity. Deacetylase assays may be conducted in a cell based or cell free format. The assays may be conducted under conditions which permit deacetylation of a substrate by the sirtuin variant. In certain embodiments, the assays are conducted in the presence of NAD⁺.

Deacetylation assay methods may involve, for example, contacting at least one acetylated sirtuin substrate with a sirtuin polypeptide in the presence of the putative sirtuin modulating compound and determining the level of acetylation of the sirtuin substrate. A change in the level of deacetylation of the substrate by the sirtuin in the presence of the putative sirtuin modulator as compared to a control (e.g., an assay without the test agent, an assay in the presence of an agent having know sirtuin modulating activity, an assay in the presence of an agent having no sirtuin modulating activity, or a value in a database) is indicative of a compound that modulates sirtuin deacetylase activity.

Putative sirtuin modulating compounds identified using the biomarker based assays described herein may be used in conjunction any type of deacetylation that assays that permits examination of sirtuin activity. For example, the putative sirtuin modulators may be used in association with a fluorescence based assay such as the assay commercially available from Biomol, e.g., the SIRT1 Fluorimetric Drug Discovery Kit (AK-555), SIRT2 Fluorimetric Drug Discovery Kit (AK-556), or SIRT3 Fluorimetric Drug Discovery Kit (AK-557) (Biomol International, Plymouth Meeting, Pa.). Other assay formats that may be used in association with the methods described herein include a nicotinamide release assay (Kaeberlein et al., J. Biol. Chem. 280(17): 17038 (2005)), a FRET assay (Marcotte et al., Anal. Biochem. 332: 90 (2004)), and a C¹⁴ NAD boron resin binding assay (McDonagh et al., Methods 36: 346 (2005)). Yet other assay formats that may be used in conjunction with the sirtuin variants described herein include radioimmunoassays (RIA), scintillation proximity assays, HPLC based assays, and reporter gene assays (e.g., for transcription factor targets). In other embodiments, the putative sirtuin modulating compounds may be used in association with a fluorescence polarization assay. Examples of fluorescence polarization assays are described herein and are also described in PCT Publication No. WO 2006/094239. In other embodiments, the putative sirtuin modulating compounds may be used in association with mass spectrometry based assays. Examples of mass spectrometry based assays are described herein and are also described in PCT Application No. PCT/US06/046021.

In various embodiments, the deacetylation assays described herein utilize a sirtuin substrate pool that comprises a plurality of copies of one or more sirtuin substrate polypeptides. In an exemplary embodiment, a sirtuin substrate pool comprises a plurality of copies of the same polypeptide substrate. Such sirtuin substrate pools may comprise the sirtuin substrate free floating in solution or attached to a solid surface such as a plate, bead, filter, etc. Combinations of free floating and anchored sirtuin substrate molecules may also be used in accordance with the methods described herein. Substrates suitable for use in accordance with the methods described herein may be based on any polypeptide that can be deacetylated by a sirtuin protein, such as, for example, p53 or histones. Exemplary substrates include, for example, the Fluor de Lys-SIRT1 substrate from BIOMOL (Plymouth Meeting, Pa.). Other suitable substrates, including for FP and mass spec based assays include, for example, Ac-EE-K(biotin)-GQSTSSHSK(Ac)NleSTEG-K(MR121)-EE-NH₂ (SEQ ID NO: 7) and Ac-EE-K(biotin)-GQSTSSHSK(Ac)NleSTEG-K(5TMR)-EE-NH₂ (SEQ ID NO: 8) wherein K(biotin) is a biotinylated lysine residue, K(Ac) is an acetylated lysine residue, Nle is norleucine, K(MR121) is a lysine residue modified by an MR121 fluorophore (excitation 635 nm/emission 680 nm), and K(5TMR) is a lysine residue modified by a 5TMR fluorophore (excitation 540 nm/emission 580 nm). The sequence of the peptide substrates are based on p53 with several modifications. In particular, all arginine and leucine residues other than the acetylated lysine residues are replaced with serine so that the peptides are not susceptible to trypsin cleavage in the absence of deacetylation. In addition, the methionine residues naturally present in the sequences are replaced with the norleucine because the methionine may be susceptible to oxidation during synthesis and purification.

In certain embodiments, the sirtuin biomarker based screening assays described herein may be used as a secondary screen to further characterize a putative sirtuin modulating compound identified, for example, using a sirtuin deacetylation assay. For example, the biomarker assays may be used to confirm that a sirtuin modulating compound identified in vitro has sirtuin modulating activity in a cellular environment, provide information about cell membrane permeability and/or cellular toxicity. Compounds that show a lower level of sirtuin modulating activity in a biomarker assay as compared to an in vitro assay may be indicative of compounds that have low cell membrane permeability or compounds that are cell membrane impermeable. Additionally, compounds that show sirtuin activating activity in an in vitro assay but show sirtuin inhibiting activity in a cell based assay may be indicative of compounds that are cytotoxic. Accordingly, such cell based assays will provide useful information for developing therapeutic agents.

Compounds that modulate sirtuin biomarker expression, which can be selected according to the methods described herein, are useful as candidate compounds for antimicrobial substances, anti-cancer agents, and a variety of other uses. For example, compounds that modulate sirtuin biomarker expression in a manner of a sirtuin activating compound may be useful for increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing, etc. In other embodiments, compounds that modulate a sirtuin biomarker in a manner similar to a sirtuin inhibiting compound may be useful for a variety of therapeutic applications including, for example, increasing cellular sensitivity to stress, increasing apoptosis, treatment of cancer, stimulation of appetite, and/or stimulation of weight gain, etc.

### 5. Kits

In other aspects, the invention provides kits for measuring the expression level of a sirtuin biomarker and screening for compounds that inhibit or enhance sirtuin activity as described above. Such kits may be useful for research purposes, drug discovery, diagnostic purposes, monitor therapeutic progress, optimizing dosage, etc.

In certain embodiments, a kit may comprise at least one component for determining the expression level of a sirtuin biomarker (as described above) and at least one sirtuin modulating compound (as described above). The biomarker-detecting component may be an antibody or an antigen-binding fragment thereof that binds to the sirtuin biomarker, a set of PCR primers that specifically amplify the sirtuin biomarker mRNA, or a solid support comprising at least a fragment of the polynucleotide sequence encoding the sirtuin biomarker attached (such as a microarray chip). The kit may further contain one or more of the following: a detection label, a positive control, a negative control, a sirtuin protein, instructions for use, a reaction vessel, buffers, etc.

In certain embodiments, a kit may comprise a cell expressing at least one sirtuin protein and at least one sirtuin biomarker (as described above) and one or more of the following: a detection label, a positive control, a negative control, instructions for use, a reaction vessel, buffers, etc.

Respective components of the kit may be combined so as to realize a final concentration that is suitable for the reaction. Further, in addition to these components, the kit may comprise a buffer that gives a condition suitable for the reaction. The sirtuin biomarker and the sirtuin protein may be combined with other components that stabilize proteins. For example, the kit components may be stored and/or shipped in the presence of about 1% BSA and about 1% polyols (e.g., sucrose or fructose) to prevent protein denaturation after lyophilization.

Also provided herein are kits for measuring the expression of the protein and RNA products of at least 1, at least 2, at least 3, at least 4, at least 5, at least 6, at least 7, at least 8, at least 9, at least 10; at least 15, at least 20, at least 25, at least 30, at least 35, at least 40, at least 45, at least 50, all or any combination of the sirtuin biomarkers. Such kits comprise materials and reagents required for measuring the expression of such protein and RNA products. In specific embodiments, the kits may further comprise one or more additional reagents employed in the various methods, such as: (1) reagents for purifying RNA from a biological sample; (2) primers for generating test nucleic acids; (3) dNTPs and/or rNTPs (either premixed or separate), optionally with one or more uniquely labeled dNTPs and/or rNTPs (e.g., biotinylated or Cy3 or Cy5 tagged dNTPs); (4) post synthesis labeling reagents, such as chemically active derivatives of fluorescent dyes; (5) enzymes, such as reverse transcriptases, DNA polymerases, and the like; (6) various buffer mediums, e.g. hybridization and washing buffers; (7) labeled probe purification reagents and components, like spin columns, etc.; (8) protein purification reagents; and (9) signal generation and detection reagents, e.g., streptavidin-alkaline phosphatase conjugate, chemifluorescent or chemiluminescent substrate, and the like. In particular embodiments, the kits comprise prelabeled quality controlled protein and or RNA isolated from a biological sample for use as a control.

In some embodiments, the kits are RT-PCR kits. In other embodiments, the kits are nucleic acid arrays and protein arrays. Such kits will at least comprise an array having associated protein or nucleic acid members that can be used to determine the expression level of sirtuin biomarkers and packaging means therefore. Alternatively the protein or nucleic acid products used to detect the expression level of sirtuin biomarkers may be prepackaged onto an array.

Each component of the kit can be provided in liquid form or dried form. Detergents, preservatives, buffers, and so on, commonly used in the art may be added to the components so long as they do not inhibit the measurement of the sirtuin deacetylase activity.

### 6. Pharmaceutical Compositions

In certain embodiments, the methods described herein may involve administration of one or more sirtuin modulating compounds to a subject. Such sirtuin modulating compounds may be known sirtuin modulating compounds or sirtuin modulating compounds identified using the methods described herein. The sirtuin-modulating compounds may be formulated in a conventional manner using one or more physiologically acceptable carriers or excipients. For example, sirtuin-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a sirtuin-modulating compound may be administered locally, at the site where the target cells are present, i.e., in a specific tissue, organ, or fluid (e.g., blood, cerebrospinal fluid, etc.).

Sirtuin-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), sirtuin-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

Sirtuin-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition, sirtuin-modulating compounds may also be formulated as a depot preparation. Such long acting formulations may be administered by implantation (for example subcutaneously or intramuscularly) or by intramuscular injection. Thus, for example, sirtuin-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

In one embodiment, a sirtuin-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Pharmaceutical compositions (including cosmetic preparations) may comprise from about 0.00001 to 100% such as from 0.001 to 10% or from 0.1% to 5% by weight of one or more sirtuin-modulating compounds described herein. In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Conditions of the eye can be treated or prevented by, e.g., systemic, topical, intraocular injection of a sirtuin-modulating compound, or by insertion of a sustained release device that releases a sirtuin-modulating compound. A sirtuin-modulating compound may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds may be injected directly into the vitreous and aqueous humour. In a further alternative, the compounds may be administered systemically, such as by intravenous infusion or injection, for treatment of the eye.

Sirtuin-modulating compounds described herein may be stored in oxygen free environment according to methods in the art. For example, resveratrol or analog thereof can be prepared in an airtight capsule for oral administration, such as Capsugel from Pfizer, Inc.

Toxicity and therapeutic efficacy of sirtuin-modulating compounds can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Sirtuin-modulating compounds that exhibit large therapeutic indexes are preferred. While sirtuin-modulating compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Preparation of Sirtuin Modulators

### 1.a Preparation of 6-(2-Nitro-phenyl)-imidazo[2,1-b]thiazole-3-carboxylic acid ethyl ester

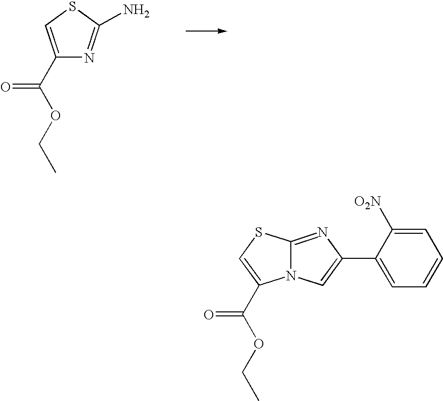

In a typical preparation, ethyl 2-aminothiazole-4-carboxylate (2.1 g, 0.0123 mol) was taken up in methyl ethyl ketone (25 mL) along with 2-bromo-2′-nitroacetophenone (3.0 g, 0.0123 mol). The reaction mixture was stirred under reflux for 18 hours. It was then cooled to room temperature and filtered to remove some of the solids. The filtrate was concentrated to afford 3.10 g of 6-(2-nitro-phenyl)-imidazo[2,1-b]thiazole-3-carboxylic acid ethyl ester (Calc'd for C₁₄H₁₂N₃O₄S: 318.3, [M+H]+ found: 319).

### 1.b Preparation of [6-(2-nitro-phenyl)-imidazo[2,1-b]thiazol-3-yl]-methanol

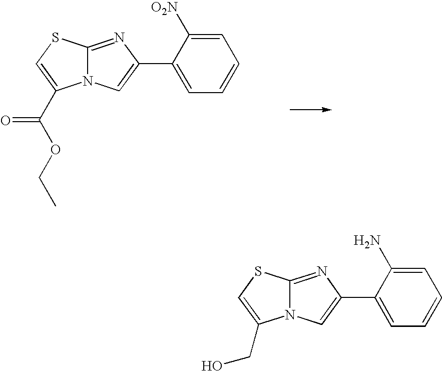

6-(2-Nitro-phenyl)-imidazo[2,1-b]thiazole-3-carboxylic acid ethyl ester (14.50 g, 0.0458 mol) was taken up in THF (100 mL) and water (100 mL) containing NaOH (7.3 g, 4 eq). The reaction mixture was stirred at room temperature for 18 hours. It was then concentrated. The aqueous layer was washed once with CH₂Cl₂ and then acidified with 6 N HCl. The solids were collected by filtration and dried to provide 7.4 g of the acid intermediate. This material (7.4 g, 0.0256 mol) was taken up in anhydrous THF (200 mL) along with N-methylmorpholine (2.8 mL, 0.0256 mol) and cooled to 0° C. Isobutyl chloroformate (3.35 mL, 0.0256 mol) was added and the reaction mixture was stirred in the ice bath for 3 hours. NaBH₄ (0.97 g, 0.0256 mol) was added as a solution in water (30 mL). The reaction mixture was stirred at 0° C. for 45 min. It was then warmed to room temperature and concentrated. The aqueous layer was extracted with CH₂Cl₂. The combined organic layers were dried (Na₂SO₄) and concentrated to afford the crude product. Purification by chromatography (Isco, using a mixture of pentane/ethyl acetate) afforded 5.20 g of [6-(2-nitro-phenyl)-imidazo[2,1-b]thiazol-3-yl]-methanol (74% yield) (Calc'd for C₁₂H₁₁N₃OS: 245.3, [M+H]+ found: 246).

### 1.c Preparation of 4-[6-(2-amino-phenyl)-imidazo[2,1-b]thiazol-3-ylmethyl]-piperazine-1-carboxylic acid tert-butyl ester

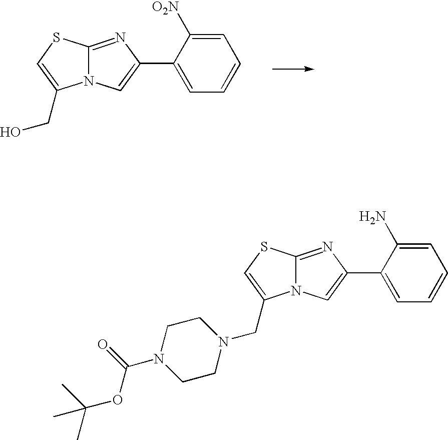

[6-(2-Nitro-phenyl)-imidazo[2,1-b]thiazol-3-yl]-methanol (1.0 g, 3.64 mmol) was dissolved in CH₂Cl₂ (100 mL) along with triethylamine (0.51 mL, 3.64 mmol). Methanesulfonyl chloride (1 eq., 0.28 mL) was added and the reaction mixture was warmed to room temperature and stirred for 15 min. It was then quenched with brine and extracted with CH₂Cl₂. The combined organic layers were dried (Na₂SO₄) and concentrated to afford the mesylate intermediate. This material was taken up CH₃CN (4 mL) along with triethylamine (0.51 mL, 3.64 mmol) and Boc-piperazine (680 mg, 3.64 mmol) and stirred at room temperature for 1 day. The reaction mixture was concentrated and the resulting residue was partitioned between CH₂Cl₂ and water. The organic layer was dried (Na₂SO₄) and concentrated to afford essentially quantitative yield of the product. This material was taken up in methanol (6 mL) and water (1 mL) along with sodium hydrosulfide hydrate (200 mg). The resulting reaction mixture was stirred under reflux for 24 hours. It was then cooled to room temperature and concentrated. The resulting residue was diluted with water (2 mL) and extracted with CH₂Cl₂. The combined organic layers were dried (Na₂SO₄) and concentrated to afford 0.90 g of 4-[6-(2-amino-phenyl)-imidazo[2,1-b]thiazol-3-ylmethyl]-piperazine-1-carboxylic acid tert-butyl ester (Calc'd for C₂₁H₂₇N₅O₂S: 413.5, [M+H]+ found: 414).

### 1.d Preparation of Compound 1

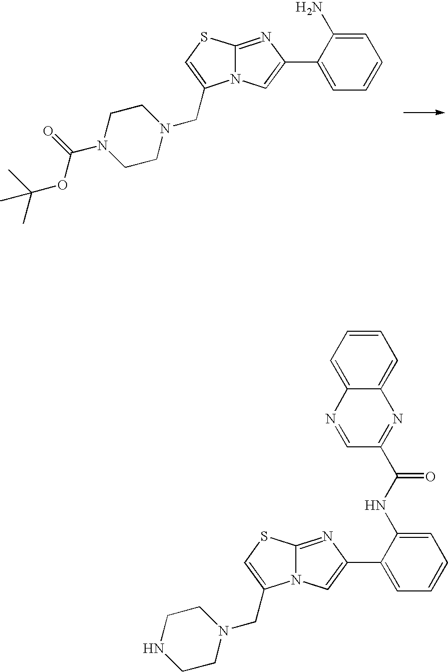

4-[6-(2-Amino-phenyl)-imidazo[2,1-b]thiazol-3-ylmethyl]-piperazine-1-carboxylic acid tert-butyl ester (0.25 mmol) was taken up in 1 mL of pyridine along with 1 eq. (50 mg) of 2-quinoxaloyl chloride. The reaction mixture was heated in a Biotage microwave reactor (at 160° C. for 10 min). It was then cooled to room temperature and concentrated. The resulting crude product was purified by chromatography (Isco, gradient elution, CH₂Cl₂ to 95% CH₂Cl₂, 4% methanol and 1% triethylamine). The purified product was then treated with a solution containing 25% trifluoroacetic acid (TFA) in CH₂Cl₂ (2 mL) for 2 hours. It was then concentrated and the resulting residue was triturated with ethyl ether to afford the desired product as the TFA salt (Calc'd for C₂₅H₂₃N₇OS: 469.5, [M+H]+ found: 470). ¹H-NMR (300 MHz, DMSO-d₆) δ: 13.9 (br s, 1H), 9.8 (br s, 1H), 9.6 (br s, 1H) 8.9-7.2 (m, 11H), 4.8 (br s, 2H). The analytical HPLC was performed on an Agilent 1100 Series HPLC equipped with a 3.5 um Eclipse XDB-C18 (4.6 mm×100 mm) column with the following conditions: acetonitrile/H₂O, modified with a 0.1% formic acid mobile phase. The gradient elution was a 5% hold (2 min), 5% to 95% gradient (11 min), 95% to 5% gradient (0.3 min), and a 5% hold (2.7 min), for a 15 min. total run time with a flow rate of 0.8 ml/min. The retention time was 3.04 min.

### 1.e Preparation of Compound 2

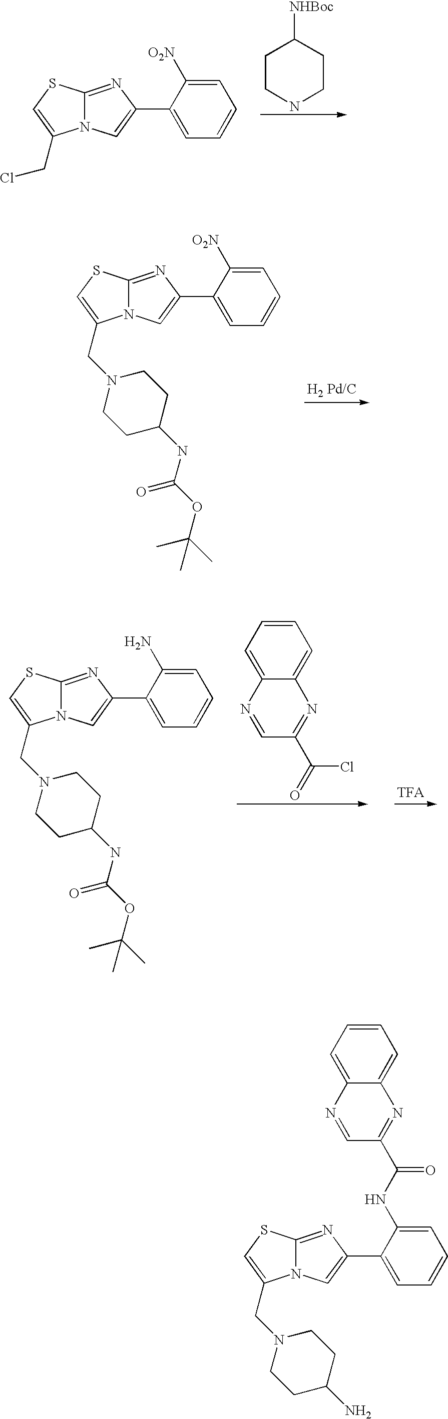

3-Chloromethyl-6-(2-nitro-phenyl)-imidazo[2,1-b]thiazole (0.200 mmol) in 3 mL of acetonitrile was neutralized with triethylamine (140 μL, 1 mmol) and tert-butyl piperidin-4-yl-carbamate (44 mg, 1.1 eq.) was added. The reaction was microwave heated at 110° C. for 30 minutes and concentrated to dryness. The residue was taken up in ethyl acetate, washed with saturated NaHCO₃, water, dried over Na₂SO₄ and concentrated to dryness to obtain {1-[6-(2-Nitro-phenyl)-imidazo[2,1-b]thiazol-3-ylmethyl]-piperidin-4-yl}-carbamic acid tert-butyl ester.

The above product was dissolved in 2:1 ethanol:tetrahydrofuran and stirred with 10% palladium on carbon (15 mg, catalytic) under hydrogen (1 atm) for 48 hours. The solution was filtered through Celite™, concentrated to dryness, and chased with CH₂Cl₂ and pentane to obtain {1-[6-(2-Amino-phenyl)-imidazo[2,1-b]thiazol-3-ylmethyl]-piperidin-4-yl}-carbamic acid tert-butyl ester as a red oil.

The above aniline was dissolved in pyridine (4 mL) and stirred with 2-quinoxaloyl chloride (46 mg, 1.2 eq.) for 18 hours at room temperature. Methanol (1 mL) was charged and the reaction mixture was concentrated to dryness. The Boc-protected product was purified on silica gel (0 to 5% methanol gradient in CH₂Cl₂), treated with 25% TFA in CH₂Cl₂ for four hours, concentrated to dryness, chased (3×) with CH₂Cl₂/pentane, and purified by preparative HPLC. The pure fractions were lyophilized in the presence of 4 N HCl (5 drops) to obtain quinoxaline-2-carboxylic acid {2-[3-(4-amino-piperidin-1-ylmethyl)-imidazo[2,1-b]thiazol-6-yl]-phenyl}-amide as a yellow solid (36.2 mg). (MS, [M⁺+H]=484.2).

### Example 2

### Identification of Sirtuin Biomarkers Using a Diet Induced Obesity Model

### 2.a Diet-Induced Obesity Model

A mouse model of diet induced obesity was used to identify sirtuin biomarkers in mice following dosing with two chemically unrelated sirtuin activators. Obesity and type II diabetes are being intensively studied in animal models, particularly the mouse. One such model is commonly referred to as the diet-induced obesity (DIO) model. Typically, C57BL/6 males are fed a high fat diet for 8 to 12 weeks and, as a result, become obese, mildly to moderately hyperglycemic, and glucose intolerant. These mice are then used to study the genetic and physiological mechanisms of obesity and type II diabetes.

51 C57BL/6 mice are started on a 60% kcal high fat diet. Mice are weighed once a week for approximately 7 weeks on the high fat diet until the average body weight of the DIO mice is 40 grams. The study is divided into 3 groups of 18 mice per group with mean average body weight/cage. Animals are dosed orally once per day as follows: Resveratrol at 1000 mg/kg in 2% HPMC/0.2% DOSS, Compound 1 at 100 mg/kg in 2% HPMC/0.2% DOSS and vehicle control animals 2% HPMC/0.2% DOSS. Concentration of compound is adjusted to proper dose according to the mean weight for each group weekly. Mice are typically dosed in the a.m. and are only dosed in the p.m. on days following a 16 hour fast. Each group is split up into 3 sub-groups for a 3, 16 and 42 day collection time point.

Once dosing starts data collections are as follow. Day 3: collect tissues, blood and glucose from 6 mice from each group 1 hour post dose. Also take fed glucose from remaining groups. Day 13: Intraperitoneal glucose tolerance test (IPGTT). Day 16: collect tissues, blood and glucose from 6 mice from each group 1 hour post dose. Also take fed glucose from remaining groups. Day 28: Fasted glucose. Day 42: Collect tissues, blood and glucose from 6 mice from each group 1 hour post dose.

### 2.b Endpoint Collection

Final blood draw for white blood cell (WBC) collection and dissection of liver, gastrocnemius muscle and epididymal white adipose tissue. Total RNA from the WBC sample and tissue samples is extracted with standard techniques (e.g. PureLink Micro-to-Midi Total RNA Purification System, Invitrogen cat. #12183-018).

### 2.c Isolation of Mouse WBC

Blood is placed in a BD® Vacutainer CPT™ Cell Preparation Tube with Sodium Citrate (BD REF 362760). The samples are centrifuged to pellet the red blood cells (RBC) at 1,700 g for 20 min. The supernatant, containing WBCs, platelets and plasma, is removed and stored on ice. The sample is then diluted with PBS and centrifuged at 300 g, for 15 min at 4° C. to pellet the WBCs. The pellet is washed one time with PBS and then the WBC pellet is resuspended in 500 uL of Freeze Media (RPMI 1640 with L-Glutamine and no phenol red+10% (final) DMSO) and stored frozen until use.

### Example 3

### Analysis of Gene Expression Levels Following Sirt1 Activation Ex Vivo

Freshly isolated human white blood cells were incubated ex vivo with two chemically unrelated Sirt1 activators and examined to determine changes in gene expression.

### 3.a Isolation of Human WBC

Approximately 6 ml of whole blood is obtained (BD® Vacutainer CPT™ Cell Preparation Tube with Sodium Heparin (BD REF 362753)), mixed by inverting and centrifuged for 20 minutes at 1700 RCF (3100 RPM) at room temperature (18-25° C.).

The plasma is removed and the cell phase, containing WBC, platelets and some plasma, is transferred to a fresh tube, diluted with PBS and centrifuged at 300 RCF (1200 RPM) for 15 minutes at room temperature (18-25° C.). The cell pellet is washed at least twice with PBS and then resuspended in 1 ml Freeze Medium (without FBS) and stored at −80° C. until use. Six milliliters of blood yields about 1 to 10 million WBC, containing about 0.4 to 4 μg total RNA, 4 to 40 μg total cell proteins and 0.015 to 0.15 ng SIRT1 protein.

### 3.b Ex Vivo Incubation of WBC

Freshly isolated WBC are resuspended in HBSS buffer (Hank's Balanced Salt Solution with calcium and magnesium but no phenol red, Invitrogen cat. #14025076). Three ml of HBSS is used for WBC isolated from 6 to 8 ml of blood, or 1 to 10 million WBC. One ml of the WBC suspension is centrifuged at 14,000 g for 5 minutes to pellet the cells. The supernatant is discarded, and the WBC pellet is frozen and kept at −80° C. until further analysis.

One ml of the WBC suspension is added to one well of a 24-well cell culture plate with a SIRT1 activator (Sirtris compound, e.g. resveratrol at 50 μM or Compound 2 at 2 uM) or vehicle (e.g. DMSO 0.25% final concentration). The cell culture plate is incubated at 37° C. and 5% CO₂ with gentle agitation for 2 hours to 20 hours. The cell suspensions are then removed (each well/sample separately) from the wells and pelleted by centrifugation at 14,000 g for 5 minutes. The pellet is frozen and kept at −80° C. until further analysis.

Total RNA from the WBC samples is extracted with standard techniques (e.g. PureLink Micro-to-Midi Total RNA Purification System, Invitrogen cat. #12183-018). The purified RNA is used to determine MCP-1 (Monocyte chemotactic protein-1) mRNA levels in the WBC exposed to a SIRT1 activator (or vehicle) with reverse transcription, real-time PCR.

### Example 4

### Gene Expression Analysis Using an Array

Total RNA isolated from either Example 2 above (mouse tissues following in vivo treatment with either Compound 1 or resveratrol) or Example 3 above (human WBC following ex vivo treatment with either Compound 2 or resveratrol) was analyzed using an Affymetrix GeneChip specific to either the mouse or human genome. Specifically, the mouse gene analysis was done at Expression Analysis Inc. (Durham, N.C.) using their Whole Transcript-Based RNA Expression Profiling service. The human gene analysis was done at the Beth Israel Deaconess Medical Center Genomics Center (Cat. #900470, Human Genome U133 Plus).

The results of the human and mouse gene chip analysis are shown in Table 1 (FIG. 1), which summarizes the results from an in vitro study using freshly isolated human WBCs and an in vivo study using the mouse model. Human WBCs were treated with resveratrol or Compound 2; mouse models were treated with resveratrol or Compound 1. Table 1 lists 43 biomarkers showing a changed expression level 2 fold or more up or down upon treatment with Compound 1 or Compound 2 and resveratrol. Specifically shown are preferred biomarkers (labeled with “***”) whose expression not only changed more than 2 fold up or down upon treatment with both compounds (i.e., Compound 1 and resveratrol with the mouse tissues or Compound 2 and resveratrol for the human WBCs) but also demonstrated the most robust or reproducible response. Preferred biomarkers either had the highest fold changes with low variability across experiments in the human WBC experiments or demonstrated the highest fold changes in tissues of interest in the mouse in vivo experiments. MCP-1 expression consistently showed a down regulation of at least 2 to 14 fold in both the human and mouse samples with all compounds tested.

Analysis of the overall pattern of change in gene expression in the 3 day liver treated with resveratrol demonstrated 1) decreased inflammatory signaling, which may suggest decreased insulin resistance; 2) increased activity of hepatic transcription factors involved in glucose homeostasis and the stress response pathway; and 3) increased activity of PGC1a and PPAR family members, which control lipid metabolism and mitochondrial biogenesis. The effects of resveratrol at three days on the liver are largely consistent with what is known in the literature about the effects of resveratrol and caloric restriction. A direct comparison of the overall pattern of change in gene expression in the 3 day liver following resveratrol or Compound 1 suggest that 1) resveratrol and Compound 1 tend to have similar consequences in the liver after three days of treatment and 2) these results are consistent with the effects of resveratrol and caloric restriction.

### Example 5

### Gene Expression Analysis Using PCR

Total RNA isolated from Example 3 above (human WBC following ex vivo treatment with either Compound 2 or resveratrol) was analyzed using reverse transcription, real-time PCR with oligo primer pairs and TaqMan probes specific for the MCP-1 gene and 18S rRNA. The levels of MCP-1 mRNA were represented relative to the levels of 18S rRNA. As compared to WBC incubated with vehicle (for 20 hours), WBC exposed to resveratrol (50 uM) had decreased MCP-1 mRNA levels by 5 to 6572-fold.

### 5.a Human MCP1 Oligo Pairs:

(SEQ ID NO: 1)

MCP1 H BP184F CAG CAG CAA GTG TCC CAA AG
and

(SEQ ID NO: 2)

MCP1 H BP278R TGG AAT CCT GAA CCC ACT TCT G.

Quantitation of the PCR signal corresponding to human MCP1 message was done using the following oligo:

(SEQ ID NO: 3)

MCP1 H Probe FAM BHQ CCACTCACCTGCTGCTACTCATTCACCA.

### 5.b Mouse MCP1 Oligos

(SEQ ID NO: 4)

MCP1 M BP85F GGC TCA GCC AGA TGC AGT TAA C
and

(SEQ ID NO: 5)

MCP1 M BP161 R GCC TAC TCA TTG GGA TCA TCT TG;

Quantitation of the PCR signal corresponding to mouse MCP1 message was done using the following oligo:

(SEQ ID NO: 6)

MCP1 M Probe FAM BHQ CCAAGGAGATCTGTGCTGACCCCAA.

### Example 6

### FGF21 Expression Analysis Using PCR

### 6.a Diet-Induced Obesity Model

As with Example 2, the diet-induced obesity (D10) model was used. 51 C57BL/6 mice are started on a 60% kcal high fat diet. Mice are weighed once a week for approximately 7 weeks on the high fat diet until the average body weight of the D10 mice is 40 grams. The study is divided into 3 groups of 18 mice per group with mean average body weight/cage. Animals are dosed orally once per day as follows: Resveratrol at 1000 mg/kg in 2% HPMC/0.2% DOSS, Compound 1 at 100 mg/kg in 2% HPMC/0.2% DOSS and vehicle control animals 2% HPMC/0.2% DOSS. Concentration of compound is adjusted to proper dose according to the mean weight for each group weekly. Mice are typically dosed in the a.m. and are only dosed in the p.m. on days following a 16 hour fast. On day 3 of dosing, liver tissue is collected from the mice 1 hour post dose.

### 6.b Endpoint Collection

The liver is isolated as in Example 2. Total RNA from the liver sample is extracted with standard techniques (e.g. PureLink Micro-to-Midi Total RNA Purification System, Invitrogen cat. #12183-018).

### 6.c Analysis of FGF21 Expression

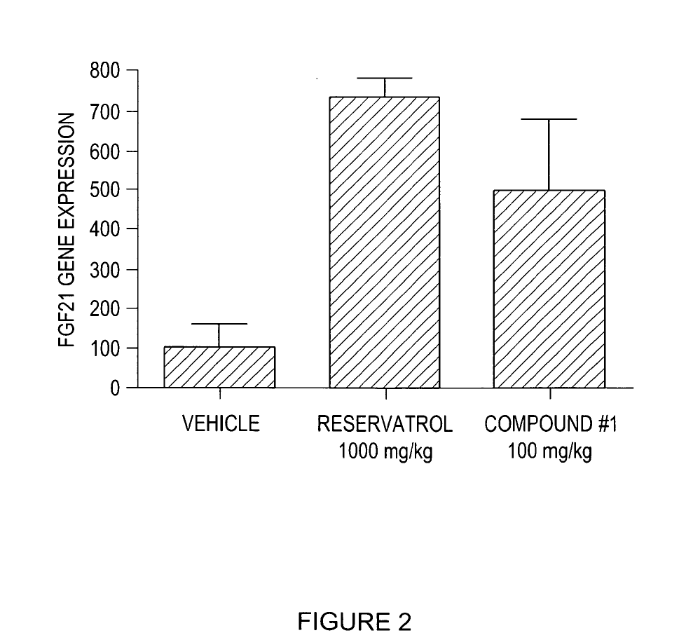

Expression of multiple genes was determine by microarray as in Example 4. FGF21 gene expression, determined in this experiment, is shown in FIG. 2. As FIG. 2 illustrates, FGF21 mRNA levels are markedly higher in resveratrol-treated and Compound 1-treated mice than control mice. This indicates that FGF21 is a biomarker of SIRT1 activity, and elevated FGF21 mRNA levels indicate elevated SIRT1 activity.

### Example 7

### SIRT1 Overexpression Results in FGF21 Upregulation

### 7.a Cell Culture

Rat H4IIE liver hepatoma cells were purchased from ATCC (#CRL-1548). Cells were maintained in DMEM supplemented with DMEM (Invitrogen #11995) with 10% Fetal Bovine Serum (low endotoxin; Benchmark; Gemini #100-106).

### 7.b Overexpression of SIRT1 in H4IIE Cells

Rat H4IIE cells were transfected using the Amaxa Nucleofector System with Kit V, using manufacturer's protocol. Plasmids transfections included 1 ug/well pCMV-GFP, 1 ug/well pCMV-SIRT1, or 5 ug/well SIRT1. Cells were plated in E-well dishes at a density of 2×10⁶ per well. 24 hours after transfection, cells were harvested and protein expression was analyzed by immunoblotting. FGF21 gene expression levels were measured by real-time PCR.

### 7.c Real-Time PCR Analysis

RNA was isolated from cell pellets using Pure Link Micro to Midi Total RNA Purification System (Invitrogen catalog #12183-018). Purified RNA was reverse-transcribed into cDNA using the High Capacity cDNA Reverse Transcription Kit (Applied Biosystems, catalog #4368813). Real time PCR reactions were performed and analyzed using Applied Biosystems 7300 Fast Real-Time PCR System.

FGF21 expression was normalized to the corresponding 18S RNA expression value from each sample. For each unique treatment, duplicate samples were generated and each sample was processed for RT-PCR in duplicate.

RT-PCR primers and probes were synthesized by Integrated DNA Technologies. Rat FGF21 probe was labeled at the 5′ end with 6-FAM™ dye (6-carboxyfluorescein) and at the 3′ end with BHQ-1 (Black Hole Quenchrer-1™). Rat 18S RNA probe was labeled at the 5′ end with JOE (6-carboxy-4′,5′-dichloro-2′, 7′-dimethoxyfluorescein) and at the 3′ end with BHQ-1.

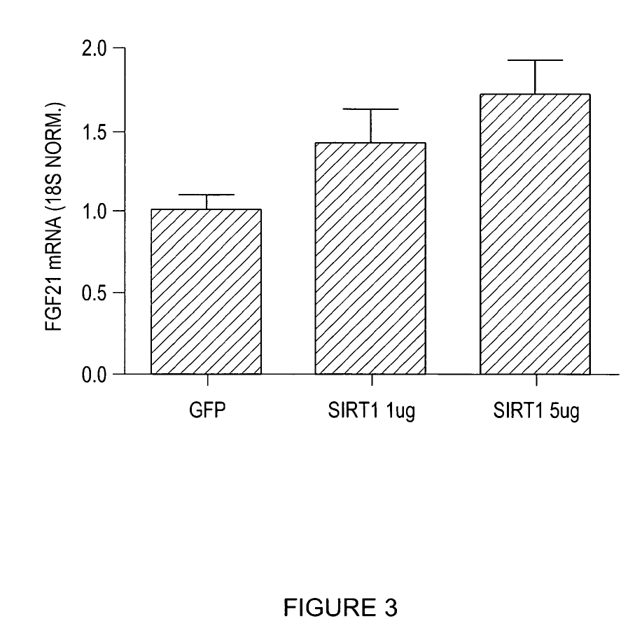

As shown in FIG. 3, FGF21 gene expression is higher in cells overexpressing SIRT1 than in control cells. This data confirms that FGF21 is a biomarker of SIRT1 activity, and that elevated FGF21 levels indicate elevated SIRT1 activity.

### 7.d Primer and Probe Sequences:

For rat FGF21:

(SEQ ID NO: 9)

| | |
|---|---|
| Probe: | CCTGCCCCTGCGTGTGCCC |

(SEQ ID NO: 10)

| | |
|---|---|
| Forward primer: | TCAGAGAGCTGCTGCTTAAGGA |

(SEQ ID NO: 11)

| | |
|---|---|
| Reverse primer: | CCCCGGGTTGCTGGAT |
| For rat 18S RNA: | |

(SEQ ID NO: 12)

| | |
|---|---|
| Forward: | CGGCTACCACATCCAAGGAA |

(SEQ ID NO: 13)

| | |
|---|---|
| Reverse: | GAGCTGGAATTACCGCGGCT |

(SEQ ID NO: 14)

| | |
|---|---|
| Probe: | TGCTGGCACCAGACTTGCCCTC |

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

1. A method of detecting sirtuin modulation in a subject comprising determining the expression level of at least one sirtuin biomarker in a biological sample from the subject, wherein a change in the expression level of the sirtuin biomarker as compared to a control is indicative of sirtuin modulation.

2. (canceled)

3. The method of claim 1, wherein the subject is suffering from a disease or disorder related to aging or stress, diabetes, obesity, a neurodegenerative disease, chemotherapeutic induced neuropathy, neuropathy associated with an ischemic event, an ocular disease or disorder, cardiovascular disease, a blood clotting disorder, inflammation, or flushing.

4-6. (canceled)

7. The method of claim 1, wherein the sirtuin biomarker is at least one of the following: MCP-1, BMP Receptor 1A, Smpd13a, CD14, ApoE, FAS, Transthyretin, FABP1, Acyl-CoA thioesterase 1, Acyl-CoA thioesterase 2, Aquaporin 4, Rrad, CXCL9, CCL8, Ppp1r3g, ApoA-I, ApoA-II, or ApoB.

8. The method of claim 7, wherein the sirtuin biomarker is MCP-1.

9. The method of claim 1, wherein the expression level of a sirtuin biomarker is determined by measuring the mRNA level of the sirtuin biomarker, the protein level of the sirtuin biomarker, or the activity of the sirtuin biomarker.

10-12. (canceled)

13. The method of claim 8, wherein a decrease in the expression level of MCP-1 as compared to a control is indicative of sirtuin activation.

14-17. (canceled)

18. A method for monitoring therapeutic treatment with a sirtuin modulator comprising determining the expression level of at least one sirtuin biomarker in a biological sample from a subject being treated with a sirtuin modulator.

19-36. (canceled)

37. A method for monitoring the progress of therapeutic treatment with a sirtuin modulator, comprising:
a) administering a sirtuin modulator to a subject,
b) obtaining a biological sample from said subject, and
c) determining the expression level of at least one sirtuin biomarker in the biological sample;
wherein an altered expression level of the sirtuin biomarker in the biological sample as compared to a control is indicative of therapeutic sirtuin modulation in said subject.

38-42. (canceled)

43. A method for identifying a subject that would benefit from treatment with a sirtuin modulating compound, comprising determining the expression level of at least one sirtuin biomarker in a biological sample from the subject, wherein an altered expression level of the sirtuin biomarker as compared to a control is indicative of a subject that would benefit from treatment with a sirtuin modulating compound.

44-48. (canceled)

49. A method for evaluating a subject's risk of developing a sirtuin-mediated disease or disorder, comprising determining the expression level of at least one sirtuin biomarker in a biological sample from the subject, wherein an altered expression level of the sirtuin biomarker as compared to a control is indicative of a subject at risk for developing a sirtuin-mediated disease or disorder.

50-52. (canceled)

53. A method for identifying a compound that modulates sirtuin activity, comprising:
a) contacting a cell expressing a sirtuin protein with a test compound, and
b) determining the expression level of at least one sirtuin biomarker in the cell, wherein a change in the expression level of the sirtuin biomarker in the presence of the test compound as compared to a control is indicative of a compound that modulates sirtuin activity.

54-56. (canceled)

57. A method for treating a sirtuin-mediated disease or disorder in a subject, comprising:
a) administering a sirtuin modulating compound to the subject, and
b) monitoring the expression level of at least one sirtuin biomarker over time to determine whether the course of treatment in the subject should be modified.

58-61. (canceled)

62. A kit for detecting the expression level of a sirtuin biomarker, comprising at least one component for determining the expression level of a sirtuin biomarker and at least one sirtuin modulating compound.

63. The kit of claim 62, wherein the component for determining the expression level of a sirtuin biomarker is at least one of the following: an antibody or an antigen-binding fragment thereof that binds to the sirtuin biomarker, a set of PCR primers that specifically amplify the sirtuin biomarker mRNA, or a solid support comprising at least a fragment of the polynucleotide sequence encoding the sirtuin biomarker attached thereto.

64. The kit of claim 62, further comprising one or more of the following: a detection label, buffer, or instructions for use.

65. The kit of claim 62, further comprising a cell line that expresses a sirtuin protein.

66. A method of determining the level of sirtuin activity in a biological sample comprising determining the expression level of at least one sirtuin biomarker in the biological sample.

67. A method of detecting sirtuin modulation in a subject comprising determining the expression level of FGF21 in a biological sample from the subject, wherein a change in the expression level of FGF21 as compared to a control is indicative of sirtuin modulation.

68. A method for monitoring therapeutic treatment with a sirtuin modulator comprising determining the expression level of FGF21 in a biological sample from a subject being treated with a sirtuin modulator, wherein a change in the expression level of FGF21 upon treatment with the sirtuin modulator is indicative of therapeutic sirtuin modulation in the subject.

69. A method for monitoring the progress of therapeutic treatment with a sirtuin modulator, comprising:
a) administering a sirtuin modulator to a subject,
b) obtaining a biological sample from said subject, and
c) determining the expression level of FGF21 in the biological sample;
wherein an altered expression level of FGF21 in the biological sample as compared to a control is indicative of therapeutic sirtuin modulation in said subject.

70. A method for identifying a subject that would benefit from treatment with a sirtuin modulating compound, comprising determining the expression level of FGF21 in a biological sample from the subject, wherein an altered expression level of FGF21 as compared to a control is indicative of a subject that would benefit from treatment with a sirtuin modulating compound.

71. A method for evaluating a subject's risk of developing a sirtuin-mediated disease or disorder, comprising determining the expression level of FGF21 in a biological sample from the subject, wherein an altered expression level of FGF21 as compared to a control is indicative of a subject at risk for developing a sirtuin-mediated disease or disorder.

72. A method for identifying a compound that modulates sirtuin activity, comprising:
a) contacting a cell expressing a sirtuin protein with a test compound, and
b) determining the expression level of FGF21 in the cell, wherein a change in the expression level of FGF21 in the presence of the test compound as compared to a control is indicative of a compound that modulates sirtuin activity.

73. A method for treating a sirtuin-mediated disease or disorder in a subject, comprising:
a) administering a sirtuin modulating compound to the subject, and
b) monitoring the expression level of FGF21 over time to determine whether the course of treatment in the subject should be modified.

74-86. (canceled)


SEQUENCE LISTING (14 sequences)

SEQ ID NO: 1 - DNA, 20 bp, Artificial Sequence
  Description of Artificial Sequence Synthetic primer
cagcagcaagtgtcccaaag

SEQ ID NO: 2 - DNA, 22 bp, Artificial Sequence
  Description of Artificial Sequence Synthetic primer
tggaatcctgaacccacttctg

SEQ ID NO: 3 - DNA, 28 bp, Artificial Sequence
  Description of Artificial Sequence Synthetic probe
ccactcacctgctgctactcattcacca

SEQ ID NO: 4 - DNA, 22 bp, Artificial Sequence
  Description of Artificial Sequence Synthetic primer
ggctcagccagatgcagttaac

SEQ ID NO: 5 - DNA, 23 bp, Artificial Sequence
  Description of Artificial Sequence Synthetic primer
gcctactcattgggatcatcttg

SEQ ID NO: 6 - DNA, 25 bp, Artificial Sequence
  Description of Artificial Sequence Synthetic probe
ccaaggagatctgtgctgaccccaa

SEQ ID NO: 7 - protein, 20 aa, Artificial Sequence
  Description of Artificial Sequence Synthetic peptide
EEKGQSTSSHSKXSTEGKEE

SEQ ID NO: 8 - protein, 20 aa, Artificial Sequence
  Description of Artificial Sequence Synthetic peptide
EEKGQSTSSHSKXSTEGKEE

SEQ ID NO: 9 - DNA, 19 bp, Artificial Sequence
  Description of Artificial Sequence Synthetic probe
cctgcccctgcgtctgccc

SEQ ID NO: 10 - DNA, 22 bp, Artificial Sequence
  Description of Artificial Sequence Synthetic primer
tcagagagctgctgcttaagga

SEQ ID NO: 11 - DNA, 16 bp, Artificial Sequence
  Description of Artificial Sequence Synthetic primer
ccccgggttgctggat

SEQ ID NO: 12 - DNA, 20 bp, Artificial Sequence
  Description of Artificial Sequence Synthetic primer
cggctaccacatccaaggaa

SEQ ID NO: 13 - DNA, 20 bp, Artificial Sequence
  Description of Artificial Sequence Synthetic primer
gagctggaattaccgcggct

SEQ ID NO: 14 - DNA, 22 bp, Artificial Sequence
  Description of Artificial Sequence Synthetic probe
tgctggcaccagacttgccctc